(19) United States
(12) Patent Application Publication
(10) Pub. No.: US 2026/0201375 A1
(43) Pub. Date: Jul. 16, 2026

(54) MODIFIED OLIGONUCLEOTIDES

(71) Applicant: Eli Lilly and Company, Indianapolis, IN (US)

(72) Inventors: Jibo Wang, Indianapolis, IN (US); Rebecca Ruth Miles, Indianapolis, IN (US); Lacie Marie Chauvigne-Hines, Indianapolis, IN (US); Suntara Cahya, Indianapolis, IN (US); Isabel C. Gonzalez Valcarcel, Indianapolis, IN (US)

(73) Assignee: Eli Lilly and Company, Indianapolis, IN (US)

(21) Appl. No.: 19/135,569

(22) PCT Filed: Dec. 4, 2023
(86) PCT No.: PCT/US2023/082237

§ 371 Date: Jun. 4, 2025

Related U.S. Application Data
(60) Provisional application No. 63/386,153, filed on Dec. 5, 2022.

(51) Int. Cl. C12N 15/113; A61K 31/712; A61K 31/7125; A61K 31/713

(52) U.S. Cl. CPC C12N 15/113; A61K 31/712; A61K 31/7125; A61K 31/713; C12N 2310/14; C12N 2310/312; C12N 2310/315; C12N 2310/321; C12N 2310/322; C12N 2310/344; C12N 2310/346; C12N 2310/3533

(57) ABSTRACT

Aspects of the disclosure relate to compositions and methods for modulating levels, function, and/or activity one or more RNA transcripts (e.g., mRNA transcripts) or the proteins in a cell or subject. The disclosure is based, in part, on chemically modified inhibitory nucleic acids that are capable of target RNA knockdown with high specificity and low off-target effects.

### RELATED APPLICATIONS

This application is a national stage filing under 35 U.S.C. § 371 of international PCT application PCT/US2023/082237, filed Dec. 4, 2023, which claims priority under 35 U.S.C. § 119(e) to U.S. provisional patent application, U.S. Ser. No. 63/386,153, filed Dec. 5, 2022, the entire contents of each of which are incorporated by reference herein.

### REFERENCE TO AN ELECTRONIC SEQUENCE LISTING

The contents of the electronic sequence listing (E058570012US01-SEQ-KZM.xml; Size: 2,820,627 bytes; and Date of Creation: Apr. 25, 2025) are herein incorporated by reference in its entirety.

### BACKGROUND

Targeted knockdown of proteins may be achieved by targeting mRNA with inhibitory nucleic acids. Chemically modified nucleic acids provide unique functional properties which enable specific targeting of mRNAs. Therapeutics comprising chemically modified oligonucleotides associated with low frequency off target knockdown effects are clinically relevant.

### SUMMARY

Aspects of the disclosure relate to chemically modified nucleic acids that bind to mRNA transcripts of target genes. In some embodiments, compositions of the disclosure are useful for treating diseases or disorders associated with dysregulated expression of mRNA and/or the protein products they encode. The disclosure is based, in part, on compositions and methods for modulating the function, activity, and/or level the protein product encoded by the target mRNA by decreasing target mRNA level and/or translation of that target mRNA in a cell or subject.

Accordingly, in some aspects, the disclosure provides a double stranded nucleic acid comprising a sense strand; and an antisense strand having a 5′-end and a 3′-end, and consisting of modified nucleotides, wherein the modified nucleotides do not comprise more than five 2′-fluoro (2′F)-modified nucleotides, and wherein the 2′F-modified nucleotides are located at positions 2, 5, 7, 14, and 16 relative to the 5′-end of the antisense strand.

In some aspects, the disclosure provides a double stranded nucleic acid comprising a sense strand; and an antisense strand having a 5′-end and a 3′-end, and consisting of modified nucleotides, wherein the modified nucleotides do not comprise more than five 2′F-modified nucleotides, and wherein the 2′F-modified nucleotides are located at positions 2, 3, 7, 14, and 16 relative to the 5′-end of the antisense strand.

In some aspects, the disclosure provides a double stranded nucleic acid comprising a sense strand; and an antisense strand having a 5′-end and a 3′-end, and consisting of modified nucleotides, wherein the modified nucleotides do not comprise more than five 2′F-modified nucleotides, and wherein the 2′F-modified nucleotides are located at positions 2, 5, 8, 14, and 16 relative to the 5′-end of the antisense strand.

In some aspects, the disclosure provides a double stranded nucleic acid comprising a sense strand; and an antisense strand having a 5′-end and a 3′-end, and consisting of modified nucleotides, wherein the modified nucleotides do not comprise more than six 2′F-modified nucleotides, and wherein the 2′F-modified nucleotides are located at positions 2, 4, 6, 8, 14, and 16 relative to the 5′-end of the antisense strand.

In some aspects, the disclosure provides a double stranded nucleic acid comprising a sense strand; and an antisense strand having a 5′-end and a 3′-end, and consisting of modified nucleotides, wherein the modified nucleotides do not comprise more than five 2′F-modified nucleotides, and wherein the 2′F-modified nucleotides are located at positions 2, 6, 8, 14, and 16 relative to the 5′-end of the antisense strand.

In some embodiments, a sense strand comprises a 5′-end and a 3′-end, and comprises 2′F-modified nucleotides at positions 9, 10, and 11 relative to the 5′-end of the sense strand; positions 7, 9, and 11 relative to the 5′-end of the sense strand; positions 7, 9, and 10 relative to the 5′-end of the sense strand; or positions 7, 10, and 11 relative to the 5′-end of the sense strand. In some embodiments, a sense strand does not comprise any other 2′F-modified nucleotides.

In some aspects, the disclosure provides a double stranded nucleic acid comprising a sense strand and an antisense strand, wherein the sense strand and the antisense strand form a duplex, wherein the antisense strand comprises a sequence complementary to a portion of a target mRNA, and wherein the antisense strand comprises five 2′F-modified nucleotides at one of the following set of positions from the 5′ end of the antisense strand: (a) positions 2, 5, 7, 14, and 16; (b) positions 2, 3, 7, 14, and 16; (c) positions 2, 5, 8, 14, and 16, and no other 2′F-modified nucleotides.

In some embodiments, a sense strand comprises three 2′F-modified nucleotides at one of the following set of positions from the 5′ end of the sense strand: (a) positions 9, 10, and 11; (b) positions 7, 9, and 11; (c) positions 7, 9, 10; or (d) positions 7, 10, and 11.

In some embodiments, an antisense strand comprises 2′-O-methyl modified nucleotides at positions other than 2′F-modified positions.

In some embodiments, a sense strand comprises 2′-O-methyl modified nucleotides or 2′-O-alkyl modified nucleotides (e.g., 2′-O—C₁₂₋₁₆ alkyl modified nucleotides) at positions other than 2′F-modified positions. In some embodiments, a sense strand comprises one or more abasic moieties at positions other than 2′F-modified positions.

In some embodiments, position 1 relative to the 5′-end of a sense strand or 5′-end of an antisense strand comprises a 5′ phosphate analog. In some embodiments, a 5′ phosphate analog comprises a 5′ vinyl phosphonate group. In some embodiments, an antisense strand comprises a 5′ phosphate analog.

In some embodiments, a sense strand is between 18 and 24 nucleotides in length. In some embodiments, a sense strand is 21 nucleotides in length.

In some embodiments, an antisense strand is between 18 and 24 nucleotides in length. In some embodiments, an antisense strand is 23 nucleotides in length.

In some embodiments, a sense strand and an antisense strand do not have the same length. In some embodiments, an antisense strand is longer than a sense strand. In some embodiments, an antisense strand is between 2 and 10 nucleotides longer than a sense strand.

In some embodiments, modified nucleotides are modified ribonucleotides. In some embodiments, modified nucleotides of a sense strand comprise one or more 2′-O-methyl (2′OMe)-modified nucleotides or one or more 2′-O-alkyl modified nucleotides (e.g., 2′-O—C₁₂₋₁₆ alkyl modified nucleotides).

In some embodiments, a double stranded nucleic acid comprises one or more abasic moieties.

In some embodiments, modified nucleotides of a sense strand comprise only 2′OMe-modified nucleotides with the exception of 2′F-modified nucleotides at listed positions.

In some embodiments, modified nucleotides of a sense strand comprise one or more 2′-O-methyl (2′OMe)-modified nucleotides, one or more 2′-O-alkyl modified nucleotides (e.g., 2′-O—C₁₂₋₁₆ alkyl modified nucleotides), or one or more abasic moieties.

In some embodiments, modified nucleotides of an antisense strand comprise only 2′OMe-modified nucleotides with the exception of 2′F-modified nucleotides at listed positions.

In some embodiments, a sense strand comprises one or more modified internucleotide linkages. In some embodiments, a sense strand comprises four modified internucleotide linkages. In some embodiments, modified internucleotide linkages comprise one or more phosphorothioate (PS) internucleotide linkages. In some embodiments, each modified internucleotide linkage of a double stranded nucleic acid sense strand is a PS internucleotide linkage.

In some embodiments, an antisense strand comprises one or more modified internucleotide linkages. In some embodiments, an antisense strand comprises four modified internucleotide linkages. In some embodiments, modified internucleotide linkages comprise one or more phosphorothioate (PS) internucleotide linkages. In some embodiments, each modified internucleotide linkage of a double stranded nucleic acid antisense strand is a PS internucleotide linkage.

In some embodiments, positions 1 and 2 (e.g., relative to the 5′ end) of a sense strand are linked by a modified internucleotide linkage. In some embodiments, positions 2 and 3 (e.g., relative to the 5′ end) of a sense strand are linked by a modified internucleotide linkage. In some embodiments, positions 1, 2, 3, and 4 (e.g., relative to the 5′ end) of a sense strand are linked by a modified internucleotide linkage. In some embodiments, the modified internucleotide linkage is a phosphorothioate (PS) internucleotide linkage.

In some embodiments, positions 1 and 2 (e.g., relative to the 5′ end) of an antisense strand are linked by a modified internucleotide linkage. In some embodiments, positions 2 and 3 (e.g., relative to the 5′ end) of an antisense strand are linked by a modified internucleotide linkage. In some embodiments, positions 1, 2, 3, and 4 (e.g., relative to the 5′ end) of an antisense strand are linked by a modified internucleotide linkage. In some embodiments, the modified internucleotide linkage is a phosphorothioate (PS) internucleotide linkage.

In some embodiments, at least two of positions 1, 2, and 3 of the 3′ end of the sense strand are linked by a modified internucleotide linkage, optionally a phosphorothioate (PS) internucleotide linkage.

In some embodiments, at least two of positions 1, 2, and 3 of the 3′ end of the antisense strand are linked by a modified internucleotide linkage, optionally a phosphorothioate (PS) internucleotide linkage.

In some embodiments, each of positions 1, 2, and 3 relative to the 3′ end of the sense strand are linked by a modified internucleotide linkage, optionally a phosphorothioate (PS) internucleotide linkage.

In some embodiments, each of positions 1, 2, and 3 relative to the 3′ end of the antisense strand are linked by a modified internucleotide linkage, optionally a phosphorothioate (PS) internucleotide linkage.

In some aspects, the disclosure provides a double stranded nucleic acid comprising a sense strand and/or antisense strand comprising the nucleotide sequence set forth in any one of SEQ ID NOs: 1-199 (e.g., as listed in Tables 1-5). In some aspects, the disclosure provides a double stranded nucleic acid comprising a sense strand and/or antisense strand comprising the nucleotide sequence and modification pattern set forth in any one of SEQ ID NOs: 1-199 (e.g., as listed in Tables 1-5). In some aspects, the disclosure provides a double stranded nucleic acid comprising a sense strand and an antisense strand comprising a SEQ ID NO selected from any one of Tables 1-5 (e.g., a sense strand comprising any one of SEQ ID NOs: 1, 2, 3, 4, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 77, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 145, 146, 152, 153, 154, 155, 156, 157, 158, 159, 160, 161, 162, 163, 164, 165, 166, 167, 168, 169, 170, 171, 172, 173, 174, or 175 and an antisense strand comprising any one of SEQ ID NOs: 5, 6, 7, 8, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 78, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 147, 148, 149, 150, 151, 176, 177, 178, 179, 180, 181, 182, 183, 184, 185, 186, 187, 188, 189, 190, 191, 192, 193, 194, 195, 196, 197, 198, or 199). In some embodiments, the double stranded nucleic acid comprises a region of complementarity with a human alpha synuclein (SNCA) mRNA transcript. In some embodiments, the double stranded nucleic acid comprises a region of complementarity with a human apolipoprotein E (APOE) mRNA transcript.

In some aspects, the disclosure provides a conjugate comprising a double stranded nucleic acid as described herein.

In some embodiments, a conjugate comprises a structure of Formula I, wherein Formula I comprises:

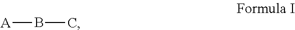

wherein “A” of Formula I comprises a double-stranded nucleic acid as described herein, “B” of Formula I comprises a bond or a linker, and “C” of Formula I comprises a delivery molecule.

In some embodiments, “B” of Formula I is connected to the 5′ end or the 3′ end of the sense strand of the double stranded nucleic acid. In some embodiments, “B” of Formula I is connected to the 3′ end of the sense strand of the double stranded nucleic acid. In some embodiments, “B” of Formula I is connected to the 5′ end or the 3′ end of the antisense strand of the double stranded nucleic acid. In some embodiments, “B” of Formula I comprises a triethylene glycol (TEG) linker. In some embodiments, “B” of Formula I comprises a linker comprising a C₆—NH₂ group. In some embodiments, “B” of Formula I does not comprise: a maleimide-methyl-tetrazine-trans-cyclo-octene (mal-tet-TCO) linker; a succinimidyl-4-(N-maleimidomethyl)cyclohexane-1-carboxylate (SMCC) linker; a linker comprising a tertiary amide which is bonded to a gem-dimethyl (GDM) group; or a linker comprising a C₆—NH₂ group.

In some embodiments, “C” of Formula I comprises one or more N-acetylgalactosamine (GalNAc) moieties. In some embodiments, “C” of Formula I comprises cholesterol. In some embodiments, “C” of Formula I comprises tocopherol.

In some embodiments, a double stranded RNA described by the disclosure does not comprise a nucleotide which is connected to: a maleimide group; a tertiary amide which is bonded to a gem-dimethyl (GDM) group; or a C₆—NH₂ group.

In some aspects, the disclosure provides a pharmaceutical composition comprising the double stranded nucleic acid as described herein, or the conjugate as described herein, and a pharmaceutically acceptable carrier.

In some aspects, the disclosure provides a method of inhibiting or reducing a target mRNA in a cell, the method comprising contacting the cell comprising the target mRNA with a double stranded nucleic acid, conjugate, or pharmaceutical composition as described herein.

In some embodiments, a cell is a mammalian cell. In some embodiments, a cell is a human cell. In some embodiments, a cell is in a subject. In some embodiments a subject is a human subject.

### DETAILED DESCRIPTION

Aspects of the disclosure relate to compositions and methods for modulating a level and/or translation of one or more RNA transcripts (e.g., mRNA transcripts) in a cell or subject. The disclosure is based, in part, on chemically modified double stranded inhibitory nucleic acids having certain patterns of 2′ Fluoro (2′F) modifications that bind to target mRNA transcripts, alter the target mRNA transcript level, and alter the activity and/or level of proteins expressed by the target mRNA transcripts. In some embodiments, the disclosure provides double stranded nucleic acids having an antisense strand comprising 2′F-modified nucleotides at the following positions relative to the 5′-end of the antisense strand: 2, 5, 7, 14, and 16; 2, 3, 7, 14, and 16; 2, 5, 8, 14, and 16; 2, 4, 6, 8, 14, and 16, or 2, 6, 8, 14, and 16. In some embodiments, the antisense strand does not comprise any other 2′F-modified nucleotides. In some embodiments, double stranded nucleic acids described herein are more durable in vivo, maintain potency, and/or have fewer off-target effects than double stranded nucleic acids that are unmodified, or have different patterns of chemical modifications.

In some embodiments, double stranded nucleic acids described by the disclosure are not conjugated to any other moiety (e.g., a delivery molecule). However, some aspects of the disclosure relate to conjugates comprising double stranded nucleic acids described herein and one or more delivery molecules.

In some embodiments, compositions of the disclosure are useful for inhibiting expression or activity of a target mRNA, and/or treating diseases or disorders associated with altered expression of specific disease-associated target mRNAs and/or proteins.

### Double Stranded Nucleic Acids

Aspects of the disclosure relate to double stranded nucleic acids. Nucleic acids comprise two or more nucleotides. As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group. A “nucleotide” can serve as a monomeric unit of nucleic acid polymers (e.g., oligonucleotides) such as deoxyribonucleic acid (DNA) and ribonucleic acid (RNA). In some embodiments, a nucleic acid is an oligonucleotide. As used herein, “oligonucleotide” means a polymer of linked nucleotides (including abasic moieties), each of which can be modified or unmodified. An oligonucleotide is typically less than about 100 nucleotides in length. Oligonucleotides may comprise unmodified DNA nucleotides, chemically modified DNA nucleotides, unmodified RNA nucleotides, chemically-modified RNA nucleotide, unnatural nucleotides (e.g., non-naturally occurring nucleotides), abasic moieties, or any combination thereof.

A double stranded nucleic acid typically forms a duplex. As used herein, a “duplex,” refers to a structure formed through complementary base pairing of two antiparallel sequences of nucleotides (i.e., in opposite directions), whether formed by two separate nucleic acid strands or by a single, folded strand (e.g., via a hairpin). As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end). In some embodiments, double stranded nucleic acids described herein form a duplex comprising a sense strand and an antisense strand. Sense strands and antisense strands of nucleotides are herein described further in the section entitled “Sense and Antisense Strands.”

The length of each strand of a double stranded nucleic acid may vary. In some embodiments, each strand of a double stranded nucleic acid ranges from about 10 nucleotides in length to about 50 nucleotides in length (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length). In some embodiments, each strand of a double stranded nucleic acid ranges from about 18 to 24 nucleotides in length. In some embodiments, a sense strand comprises or consists of 21 nucleotides. In some embodiments, an antisense strand comprises or consists of 23 nucleotides. It should be appreciated that although the foregoing numeric ranges refer to “nucleotides”, the disclosure also contemplates abasic moieties to be included in such ranges.

In some embodiments, the sense strand and antisense strand of a double stranded nucleic acid are the same length (e.g., the double stranded nucleic acid is “blunt-ended”). In some embodiments, the sense strand is longer than the antisense strand. In some embodiments, the antisense strand is longer than the sense strand. In some embodiments, the sense strand and antisense strand are of different lengths, and the double stranded nucleic acid comprises one or two overhangs. As used herein, “overhang” means the unpaired nucleotide or nucleotides that protrude from the duplex structure of a double stranded oligonucleotide. An overhang may include one or more unpaired nucleotides extending from a duplex region at the 5′ terminus or 3′ terminus of a double stranded oligonucleotide. The overhang can be a 3′ or 5′ overhang on the antisense strand or sense strand of a double stranded oligonucleotide. In some embodiments, the sense strand is longer than the antisense strand by 1-10 nucleotides (e.g., 1, 2, 3, 4, 5, 6, 7, 8, 9, or 10 nucleotides). In some embodiments, the antisense strand is longer than the sense strand by 1-10 nucleotides (e.g., 1, 2, 3, 4, 5, 6, 7, 8, 9, or 10 nucleotides). In some embodiments, a double stranded nucleic acid comprises one overhang on the sense strand (e.g., a 5′ sequence on the sense strand that extends past the 5′-most nucleotide of the antisense strand). In some embodiments, a double stranded nucleic acid comprises one overhang on the antisense strand (e.g., a 5′ sequence on the antisense strand that extends past the 5′-most nucleotide of the sense strand). In some embodiments, a double stranded nucleic acid comprises two overhangs on the sense strand. In some embodiments, a double stranded nucleic acid comprises two overhangs on the antisense strand. In some embodiments, a double stranded nucleic acid comprises one overhang on the sense strand and one overhang on the antisense strand. In some embodiments, an overhang sequence is about 2, 3, 4, 5, 6, 7, 8, 9 or 10 nucleotides in length.

In some embodiments, a double stranded nucleic acid is an inhibitory nucleic acid. As used herein, an “inhibitory nucleic acid” refers to an oligonucleotide capable of decreasing the level, function, and/or activity of an mRNA transcript (e.g., a target mRNA transcript) or the protein encoded by the mRNA inside of a cell. As used herein, a “target mRNA” is an mRNA encoding a protein associated with a disease or disorder, including but not limited to mRNAs encoding mutant proteins, pathogenic protein isoforms, or mRNAs that are not normally expressed in healthy cells. In some embodiments, a target mRNA is a wild-type mRNA (e.g., an mRNA encoding a wild-type protein). In some embodiments, inhibitory nucleic acids may be used to cleave target mRNA or inhibit translation of a target mRNA that comprises one or more mutations (e.g., substitutions, deletions, and/or insertions) relative to the wild-type version of the sequence found in nature. In some embodiments, inhibitory nucleic acids may be used to inhibit translation of a target mRNA that code for shortened versions of genes that are commonly found in nature (e.g., truncation mutants or mutants lacking catalytic and/or regulatory domains).

In some embodiments, the antisense strand of a double stranded nucleic acid hybridizes with a target mRNA molecule. Hybridization of nucleic acids typically comprises binding of one nucleic acid strand to a complementary region on another nucleic acid strand. The terms “bind” and “binds” as used herein are intended to mean, unless indicated otherwise, the ability of a molecule to form a chemical bond or attractive interaction with another molecule, which results in proximity of the two molecules as determined by common methods known in the art. As used herein, “complementary” means a structural relationship between two nucleotides (e.g., on two opposing nucleic acids or on opposing regions of a single nucleic acid strand, e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

In some embodiments, a target mRNA comprises a degree of sequence identity relative to the sense strand that is sufficient to promote specific binding (e.g., hybridization) by the antisense strand of a double stranded nucleic acid (e.g., an inhibitory nucleic acid). The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Megalign (DNASTAR), Clustal W2.0 or Clustal X2.0 software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

In some embodiments, a strand of a double stranded nucleic acid (e.g., sense strand) comprises between about 80% and 100% (e.g., about 80%, 85%, 90%, 95%, 99%, 99.9%, or 100%) sequence identity with a target mRNA. In some embodiments, a strand of a double stranded nucleic acid (e.g., sense strand) has 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a target mRNA. In some embodiments, an inhibitory nucleic acid comprises an antisense strand comprising a region of complementarity that is at least 80% complementary to a portion of a target mRNA (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the nucleotides of the antisense strand hybridize to the ribonucleotides of the target mRNA). In some embodiments, an inhibitory nucleic acid comprises an antisense strand comprising a region of complementarity that is perfectly complementary to a portion of a target mRNA (e.g., 100% of the nucleotides of the antisense strand hybridize to the ribonucleotides of the target mRNA).

In some embodiments, a double stranded inhibitory nucleic acid comprises a single oligonucleotide comprising sense and antisense strands linked by a linker, a hairpin loop, and/or a common backbone which intramolecularly hybridizes via nucleotides in the sense and antisense strands. Such a double stranded inhibitory nucleic acid would comprise a single 5′ and 3′ end and a loop region located between the two sense and antisense sequences that do not form base pairs with any other nucleotides within the inhibitory nucleic acid. In some embodiments, a double stranded inhibitory nucleic acid comprises two complementary oligonucleotides having respective 5′ and 3′ ends and respective backbones. In some embodiments, inhibitory nucleic acids are double stranded RNAs (dsRNAs). In some embodiments, inhibitory nucleic acids that are dsRNAs function like RNA interference (RNAi) molecules and thus may be referred to as RNAi agents. In some embodiments, inhibitory nucleic acids that are dsRNAs function like small interfering RNA molecules and thus may be referred to as siRNAs. The term “knockdown” or “expression knockdown” refers to reduced mRNA or protein expression of a gene after treatment with an inhibitory nucleic acid (e.g., RNAi agent).

In some embodiments, the double stranded nucleic acid is a RNAi agent. As used herein, “RNAi agent”, “iRNA,” “iRNA agent,” “RNAi,” and “RNA interference agent” means an agent that contains ribonucleotides or RNA and mediates the targeted cleavage of a RNA transcript via RNA interference, e.g., through a RNA-induced silencing complex (RISC) pathway. In some embodiments, the RNAi agent comprises a sense strand and an antisense strand, and the sense strand and the antisense strand form a duplex. In some embodiments, the sense strand and antisense strand of the RNAi agent are 21-23 nucleotides in length.

To provide desirable characteristics, for example to minimize the likelihood of off-target effects, to increase potency, and/or to increase durability, a double stranded nucleic acid may be designed to ensure that it does not have a sequence (e.g., of 5 or more consecutive nucleotides) that is complementary with an off-target nucleic acid (e.g., an mRNA that does not comprise the sequence of the target mRNA), or engineered to comprise one or more modified nucleotides that reduce or prevent off-target interactions or degradation (e.g., enzymatic cleavage). Aspects of the disclosure relate to double stranded nucleic acids having certain patterns of 2′F-modifications that reduce promiscuity (e.g., off-target binding) of the nucleic acids relative to double stranded nucleic acids not having modifications or not having the same patterns of 2′F-modifications described herein.

Aspects of the disclosure relate to double stranded nucleic acids comprising certain patterns of chemical modifications (e.g., modified nucleotides, abasic moieties, modified internucleotide linkages, etc.). As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide.

Chemical modifications may be used to endow a double stranded nucleic acid with specific functional characteristics not exhibited by unmodified nucleic acids. Double stranded nucleic acids of the disclosure may be modified to achieve one or more desired properties, such as, for example, improved cellular uptake, improved stability, reduced immunogenicity, improved potency, improved target hybridization, susceptibility to RNAse cleavage, etc. In some embodiments, an inhibitory nucleic acid is modified such that when present in a cell that contains an mRNA target, it is capable of hybridizing with the mRNA transcribed from the DNA sequence and inducing cleavage of the mRNA.

A modified nucleotide can have, for example, one or more chemical modifications in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, or 2′-O-alkyl modified nucleotide, e.g., a 2′O—C₁₂₋₁₆ alkyl modified nucleotide. As used herein, the term “alkyl” means saturated linear or branched-chain monovalent hydrocarbon radical, containing the indicated number of carbon atoms. For example, “C₁₂₋₁₆ alkyl” means a radical having 12-16 carbon atoms in a linear or branched arrangement. Additional examples of nucleotides comprising modifications to a 2′ carbon of the sugar group include but are not limited to D-ribose, 2′-deoxy, 2′-O-alkyl (including 2′-O-methyl and 2′-O-ethyl), 2′-alkoxy, 2′-amino, 2′-aminoalkoxy, 2′-S-alkyl, 2′-2-O-methoxyethoxy, 2′-O-methoxyethyl, 2′-allyloxy (OCH2CH═CH2), 2′-propargyl, 2′-propyl, ethynyl, ethenyl, propenyl, and cyano and the like. In some embodiments, the 2′-modified nucleotide comprises a 2′-O-4′-C methylene bridge, such as a locked inhibitory nucleic acid (LNA) nucleotide. In some embodiments, the 2′-hydroxyl group is linked to the 3′ or 4′ carbon atom of the sugar ring thereby forming a bicyclic sugar moiety. Other 2′ modifications are found in the art.

In some embodiments, the modified nucleotide has a phosphate analog. As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. A 5′ phosphate analog can include a phosphatase-resistant linkage. Examples of phosphate analogs include 5′ methylene phosphonate (5′-MP) and 5′-(E)-vinyl phosphonate (5′-VP). In some embodiments, a phosphate analog is positioned at position 1 relative to the sense strand or the antisense strand of a double stranded RNA. In some embodiments, the phosphate analog is 5′-VP. In some embodiments, the antisense strand comprises the 5′ phosphate analog.

A double stranded nucleic acid may comprise one or more abasic moieties or inverted abasic moieties. As used herein, “abasic moiety” or “apurinic/apyrimidic site” refers to a molecule derived from a nucleotide which comprises a ribose (or deoxyribose) sugar and phosphate bond from which a nitrogenous base (e.g., nucleobase) has been removed by cleaving the glycosidic bond between the ribose (or deoxyribose) sugar and the base.

In some embodiments, a double stranded nucleic acid comprises one or more (e.g., 1, 2, 3, 4, 5, or more) modified internucleotide linkages. As used herein, “modified internucleotide linkage” refers to an internucleotide linkage having one or more chemical modifications when compared with a reference internucleotide linkage having a phosphodiester bond. A modified internucleotide linkage can be a non-naturally occurring linkage. In some embodiments, the modified internucleotide linkage is phosphorothioate (PS) linkage. Examples of other modified nucleic acids and modified internucleotide linkages include, but are not limited to, borano-phosphate, alkyl phosphonate inhibitory nucleic acids, peptide inhibitory nucleic acids, and morpholinos. Morpholino backbones are described, for example by Corey and Abrams Genome Biol. 2001; 2(5): reviews1015.1-reviews1015.3.

The number of modified internucleotide linkages in each strand of a double stranded nucleic acid may vary. In some embodiments, each strand of a double stranded nucleic acid comprises between 1 and 23 (e.g., 1, 2, 3, 4, 5, 6, 7, 8, 9, 10, 11, 12, 13, 14, 15, 16, 17, 18, 19, 20, 21, 22, or 23) modified internucleotide linkages. In some embodiments, a sense strand of a double stranded nucleic acid comprises between 1 and 5 (e.g., 1, 2, 3, 4, or 5) modified internucleotide linkages. In some embodiments, the modified internucleotide linkages are positioned beginning at the first nucleotide of the 5′-end of the sense strand. In some embodiments, the modified internucleotide linkages are positioned between the last two nucleotides of the 3′-end of the sense strand (e.g., at one or more of positions 1, 2, and 3 relative to the 3′ end of the sense strand). In some embodiments, an antisense strand of a double stranded nucleic acid comprises between 1 and 5 (e.g., 1, 2, 3, 4, or 5) modified internucleotide linkages. In some embodiments, the modified internucleotide linkages are positioned beginning at the first nucleotide of the 5′-end of the antisense strand. In some embodiments, the modified internucleotide linkages are positioned between the last two nucleotides of the 3′-end of the antisense strand (e.g., at one or more of positions 1, 2, and 3 relative to the 3′ end of the antisense strand). In some embodiments, each of the modified internucleotide linkages is a phosphorothioate (PS) linkage.

In some embodiments, the antisense strand of a double stranded nucleic acid comprises no more than four phosphorothioate internucleotide linkages. In some embodiments, positions 1 and 2 (e.g., relative to the 5′ end or 3′ end) of a sense strand are linked by modified internucleotide linkages. In some embodiments, positions 1 and 2 (e.g., relative to the 5′ end or 3′ end) of an antisense strand are linked by a phosphorothioate internucleotide linkage. In some embodiments, positions 2 and 3 (e.g., relative to the 5′ end or 3′ end) of a sense strand are linked by a modified internucleotide linkage. In some embodiments, positions 2 and 3 (e.g., relative to the 5′ end or 3′ end) of an antisense strand are linked by a phosphorothioate internucleotide linkage.

In some embodiments, the target mRNA of an inhibitory nucleic acid corresponds to a gene sequence encoding a wild-type protein, or any variant (e.g., mutant, disease-associated allele, isoform, etc.) thereof. However, these examples should be considered non-limiting as the chemical modifications described in the present disclosure may be applied to virtually any inhibitory nucleic acid sequence to perform targeted knockdown of target mRNAs.

In some embodiments, a double stranded nucleic acid downregulates expression or activity of a target mRNA. The amount of downregulation mediated by a double stranded nucleic acid may vary. In some embodiments, a double stranded nucleic acid decreases expression level or activity of a target mRNA transcript between 1-fold and 100-fold, 2-fold and 10-fold, 5-fold and 20-fold, 10-fold and 30-fold, 20-fold and 50-fold, or 25-fold and 100-fold, or any value therebetween (e.g., relative to the expression or activity of the target mRNA that has not been contacted with the double stranded nucleic acid). In some embodiments, a double stranded nucleic acid decreases expression level or activity of a target mRNA transcript by about 2-fold, 3-fold, 4-fold, 5-fold, 6-fold, 7-fold, 8-fold, 9-fold, 10-fold, 15-fold, or 20-fold (e.g., relative to the expression or activity of the target mRNA that has not been contacted with the double stranded nucleic acid). In some embodiments, a double stranded nucleic acid decreases expression level or activity of a target mRNA transcript more than 100-fold, for example at least 200-fold, 400-fold, 500-fold, or 1000-fold (e.g., relative to the expression or activity of the target mRNA that has not been contacted with the double stranded nucleic acid).

In some embodiments, a double stranded nucleic acid is an isolated nucleic acid. It will be understood by those of skill in the art that an “isolated” nucleic acid is one that is artificially produced. Artificial production of an isolated nucleic acid may be achieved, for example, through amplification in vitro through polymerase chain reaction (PCR), in vitro transcription, in vitro reverse transcription, recombinant cloning, or chemical synthesis. Methods of synthesizing isolated nucleic acids, for example RNAs, are known in the art, for example as described by Soukchareun et al. Preparation and characterization of antisense oligonucleotide-peptide hybrids containing viral fusion peptides. Bioconjug Chem. 1995 January-February; 6(1):43-53. doi: 10.1021/bc00031a004. PMID: 7711103. In some embodiments, sense strand and antisense strands of inhibitory nucleic acids can be synthesized using, for example, solid-phase synthesis by employing phosphoramidite chemistry methodology (e.g., Current Protocols in Inhibitory nucleic acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT ((dimethylaminomethylidene) amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a double stranded RNA.

### Sense and Antisense Strands

Aspects of the disclosure relate to double stranded nucleic acids having sense and antisense strands, each of which comprise certain patterns of chemical modifications (e.g., patterns of 2′F-modified nucleotides). As used herein, “antisense strand” means a single-stranded oligonucleotide that is complementary to a region of a target sequence (e.g., a sequence of a target mRNA). Likewise, and as used herein, “sense strand” means a single-stranded oligonucleotide that is complementary to a region of an antisense strand.

Aspects of the disclosure are based, in part, on antisense strands having 2′F-modified nucleotides at the following combinations of positions relative to the 5′ end of the antisense strand:2, 5, 7, 14, and 16; 2, 3, 7, 14, and 16; 2, 5, 8, 14, and 16; 2, 4, 6, 8, 14, and 16; or 2, 6, 8, 14, and 16. In some embodiments, the 2′F-modified antisense strands do not comprise any 2′F-modified nucleotides other than those at the listed positions. In some embodiments antisense strands (and double stranded nucleic acids comprising such antisense strands) have improved potency, durability, and/or reduced off-target effects relative to previously described chemically-modified double stranded nucleic acids.

In some embodiments, an antisense strand comprises 2′F-modified nucleotides at the following positions relative to the 5′ end of the antisense strand: 2, 5, 7, 14, and 16, and does not comprise any 2′F-modified nucleotides other than those at the listed positions. In some embodiments, the antisense strand further comprises one or more additional modified nucleotides. In some embodiments, the antisense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification, e.g., 2′O-methyl (2′O-Me) modification or 2′O—C₁₂₋₁₆ alkyl modification. In some embodiments, each of the one or more additional modified nucleotides comprises a 2′O-methyl (2′O-Me) modification. In some embodiments, the antisense strand comprises one or more abasic moieties. In some embodiments, the antisense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, an antisense strand comprises PS linkages between positions 1 and 2 relative to the 5′ end of the antisense strand. In some embodiments, an antisense strand comprises PS linkages between positions 1 and 2 relative to the 3′ end of the antisense strand. In some embodiments, an antisense strand comprises PS linkages between positions 1 and 2 relative to the 5′ end of the antisense strand, and positions 1 and 2 relative to the 3′ end of the antisense strand. In some embodiments, an antisense strand comprises PS linkages between positions 1 and 2, and positions 2 and 3, relative to the 3′ end of the antisense strand. In some embodiments, an antisense strand comprises PS linkages between positions 1 and 2, and positions 2 and 3, relative to the 5′ end of the antisense strand, and positions 1 and 2, and positions 2 and 3, relative to the 3′ end of the antisense strand.

In some embodiments, the antisense strand is hybridized to a sense strand. In some embodiments, the sense strand comprises one or more 2′F-modified nucleotides. In some embodiments, the sense strand comprises 2′F-modified nucleotides at the following positions: (a) positions 9, 10, and 11; (b) positions 7, 9, and 11; (c) positions 7, 9, 10; or (d) positions 7, 10, and 11. In some embodiments, the sense strand does not comprise any 2′F-modified nucleotides at positions other than the listed positions. In some embodiments, the sense strand further comprises one or more additional modified nucleotides. In some embodiments, the sense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the sense strand comprises one or more abasic moieties. In some embodiments, the sense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, a sense strand comprises PS linkages between positions 1 and 2 relative to the 5′ end of the sense strand. In some embodiments, a sense strand comprises PS linkages between positions 1 and 2 relative to the 3′ end of the sense strand. In some embodiments, a sense strand comprises PS linkages between positions 1 and 2 relative to the 5′ end of the sense strand, and positions 1 and 2 relative to the 3′ end of the sense strand. In some embodiments, a sense strand comprises PS linkages between positions 1 and 2, and positions 2 and 3, relative to the 3′ end of the sense strand. In some embodiments, a sense strand comprises PS linkages between positions 1 and 2, and positions 2 and 3, relative to the 5′ end of the sense strand, and positions 1 and 2, and positions 2 and 3, relative to the 3′ end of the sense strand.

In some embodiments, the disclosure provides a double stranded nucleic acid comprising a sense strand; and an antisense strand having a 5′-end and a 3′-end, and consisting of modified nucleotides, wherein the modified nucleotides consist of 2′F-modified nucleotides located at positions 2, 3, 7, 14, and 16 relative to the 5′-end of the antisense strand, and 2′O-Me modified nucleotides at the other positions of the antisense strand, and wherein the sense strand comprises 2′F-modified nucleotides located at and only at the following positions: (a) positions 9, 10, and 11; (b) positions 7, 9, and 11; (c) positions 7, 9, 10; or (d) positions 7, 10, and 11, and 2′O-Me modified nucleotides, 2′O—C₁₂₋₁₆ alkyl modified nucleotides or abasic moiety at the other positions of the sense strand; and wherein each of the sense and antisense strands comprise PS linkages between positions 1 and 2, and positions 2 and 3, relative to their respective 5′ ends and 3′ ends.

In some embodiments, an antisense strand comprises 2′F-modified nucleotides at the following positions relative to the 5′ end of the antisense strand: 2, 3, 7, 14, and 16, and does not comprise any 2′F-modified nucleotides other than those at the listed positions. In some embodiments, the antisense strand further comprises one or more additional modified nucleotides. In some embodiments, the antisense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the antisense strand comprises one or more abasic moieties. In some embodiments, the antisense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, an antisense strand comprises PS linkages between positions 1 and 2 relative to the 5′ end of the antisense strand. In some embodiments, an antisense strand comprises PS linkages between positions 1 and 2 relative to the 3′ end of the antisense strand. In some embodiments, an antisense strand comprises PS linkages between positions 1 and 2 relative to the 5′ end of the antisense strand, and positions 1 and 2 relative to the 3′ end of the antisense strand. In some embodiments, an antisense strand comprises PS linkages between positions 1 and 2, and positions 2 and 3, relative to the 3′ end of the antisense strand. In some embodiments, an antisense strand comprises PS linkages between positions 1 and 2, and positions 2 and 3, relative to the 5′ end of the antisense strand, and positions 1 and 2, and positions 2 and 3, relative to the 3′ end of the antisense strand.

In some embodiments, the antisense strand is hybridized to a sense strand. In some embodiments, the sense strand comprises one or more 2′F-modified nucleotides. In some embodiments, the sense strand comprises 2′F-modified nucleotides at the following positions: (a) positions 9, 10, and 11; (b) positions 7, 9, and 11; (c) positions 7, 9, 10; or (d) positions 7, 10, and 11. In some embodiments, the sense strand does not comprise any 2′F-modified nucleotides at positions other than the listed positions. In some embodiments, the sense strand further comprises one or more additional modified nucleotides. In some embodiments, the sense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the sense strand comprises one or more 2′O—C₁₂₋₁₆ alkyl modified nucleotides. In some embodiments, the sense strand comprises one or more abasic moieties. In some embodiments, the sense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, a sense strand comprises PS linkages between positions 1 and 2 relative to the 5′ end of the sense strand. In some embodiments, a sense strand comprises PS linkages between positions 1 and 2 relative to the 3′ end of the sense strand. In some embodiments, a sense strand comprises PS linkages between positions 1 and 2 relative to the 5′ end of the sense strand, and positions 1 and 2 relative to the 3′ end of the sense strand. In some embodiments, a sense strand comprises PS linkages between positions 1 and 2, and positions 2 and 3, relative to the 3′ end of the sense strand. In some embodiments, a sense strand comprises PS linkages between positions 1 and 2, and positions 2 and 3, relative to the 5′ end of the sense strand, and positions 1 and 2, and positions 2 and 3, relative to the 3′ end of the sense strand.

In some embodiments, the disclosure provides a double stranded nucleic acid comprising a sense strand; and an antisense strand having a 5′-end and a 3′-end, and consisting of modified nucleotides, wherein the modified nucleotides consist of 2′F-modified nucleotides located at positions 2, 5, 7, 14, and 16 relative to the 5′-end of the antisense strand, and 2′O-Me modified nucleotides at the other positions of the antisense strand, and wherein the sense strand comprises 2′F-modified nucleotides located at and only at the following positions: (a) positions 9, 10, and 11; (b) positions 7, 9, and 11; (c) positions 7, 9, 10; or (d) positions 7, 10, and 11, and 2′O-Me modified nucleotides, 2′O—C₁₂₋₁₆ alkyl modified nucleotides or abasic moiety at the other positions of the sense strand; and wherein each of the sense and antisense strands comprise PS linkages at between positions 1 and 2, and positions 2 and 3, relative to their respective 5′ ends and 3′ ends.

In some embodiments, an antisense strand comprises 2′F-modified nucleotides at the following positions relative to the 5′ end of the antisense strand: 2, 5, 7, 14, and 16, and does not comprise any 2′F-modified nucleotides other than those at the listed positions. In some embodiments, the antisense strand further comprises one or more additional modified nucleotides. In some embodiments, the antisense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the antisense strand comprises one or more abasic moieties. In some embodiments, the antisense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, an antisense strand comprises PS linkages between positions 1 and 2 relative to the 5′ end of the antisense strand. In some embodiments, an antisense strand comprises PS linkages between positions 1 and 2 relative to the 3′ end of the antisense strand. In some embodiments, an antisense strand comprises PS linkages between positions 1 and 2 relative to the 5′ end of the antisense strand, and positions 1 and 2 relative to the 3′ end of the antisense strand. In some embodiments, an antisense strand comprises PS linkages between positions 1 and 2, and positions 2 and 3, relative to the 3′ end of the antisense strand. In some embodiments, an antisense strand comprises PS linkages between positions 1 and 2, and positions 2 and 3, relative to the 5′ end of the antisense strand, and positions 1 and 2, and positions 2 and 3, relative to the 3′ end of the antisense strand.

In some embodiments, the antisense strand is hybridized to a sense strand. In some embodiments, the sense strand comprises one or more 2′F-modified nucleotides. In some embodiments, the sense strand comprises 2′F-modified nucleotides at the following positions: (a) positions 9, 10, and 11; (b) positions 7, 9, and 11; (c) positions 7, 9, 10; or (d) positions 7, 10, and 11. In some embodiments, the sense strand does not comprise any 2′F-modified nucleotides at positions other than the listed positions. In some embodiments, the sense strand further comprises one or more additional modified nucleotides. In some embodiments, the sense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the sense strand comprises one or more 2′O—C₁₂₋₁₆ alkyl modified nucleotides. In some embodiments, the sense strand comprises one or more abasic moieties. In some embodiments, the sense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, a sense strand comprises PS linkages between positions 1 and 2 relative to the 5′ end of the sense strand. In some embodiments, a sense strand comprises PS linkages between positions 1 and 2 relative to the 3′ end of the sense strand. In some embodiments, a sense strand comprises PS linkages between positions 1 and 2 relative to the 5′ end of the sense strand, and positions 1 and 2 relative to the 3′ end of the sense strand. In some embodiments, a sense strand comprises PS linkages between positions 1 and 2, and positions 2 and 3, relative to the 3′ end of the sense strand. In some embodiments, a sense strand comprises PS linkages between positions 1 and 2, and positions 2 and 3, relative to the 5′ end of the sense strand, and positions 1 and 2, and positions 2 and 3, relative to the 3′ end of the sense strand.

In some embodiments, the disclosure provides a double stranded nucleic acid comprising a sense strand; and an antisense strand having a 5′-end and a 3′-end, and consisting of modified nucleotides, wherein the modified nucleotides consist of 2′F-modified nucleotides located at positions 2, 5, 8, 14, and 16 relative to the 5′-end of the antisense strand, and 2′O-Me modified nucleotides at the other positions of the antisense strand, and wherein the sense strand comprises 2′F-modified nucleotides located at and only at the following positions: (a) positions 9, 10, and 11; (b) positions 7, 9, and 11; (c) positions 7, 9, 10; or (d) positions 7, 10, and 11, and 2′O-Me modified nucleotides, 2′O—C₁₂₋₁₆ alkyl modified nucleotides or abasic moiety at the other positions of the sense strand; and wherein each of the sense and antisense strands comprise PS linkages at between positions 1 and 2, and positions 2 and 3, relative to their respective 5′ ends and 3′ ends.

In some embodiments, an antisense strand comprises 2′F-modified nucleotides at the following positions relative to the 5′ end of the antisense strand: 2, 5, 8, 14, and 16, and does not comprise any 2′F-modified nucleotides other than those at the listed positions. In some embodiments, the antisense strand further comprises one or more additional modified nucleotides. In some embodiments, the antisense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the antisense strand comprises one or more abasic moieties. In some embodiments, the antisense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, an antisense strand comprises PS linkages between positions 1 and 2 relative to the 5′ end of the antisense strand. In some embodiments, an antisense strand comprises PS linkages between positions 1 and 2 relative to the 3′ end of the antisense strand. In some embodiments, an antisense strand comprises PS linkages between positions 1 and 2 relative to the 5′ end of the antisense strand, and positions 1 and 2 relative to the 3′ end of the antisense strand. In some embodiments, an antisense strand comprises PS linkages between positions 1 and 2, and positions 2 and 3, relative to the 3′ end of the antisense strand. In some embodiments, an antisense strand comprises PS linkages between positions 1 and 2, and positions 2 and 3, relative to the 5′ end of the antisense strand, and positions 1 and 2, and positions 2 and 3, relative to the 3′ end of the antisense strand.

In some embodiments, the antisense strand is hybridized to a sense strand. In some embodiments, the sense strand comprises one or more 2′F-modified nucleotides. In some embodiments, the sense strand comprises 2′F-modified nucleotides at the following positions: (a) positions 9, 10, and 11; (b) positions 7, 9, and 11; (c) positions 7, 9, 10; or (d) positions 7, 10, and 11. In some embodiments, the sense strand does not comprise any 2′F-modified nucleotides at positions other than the listed positions. In some embodiments, the sense strand further comprises one or more additional modified nucleotides. In some embodiments, the sense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the sense strand comprises one or more 2′O—C₁₂₋₁₆ alkyl modified nucleotides. In some embodiments, the sense strand comprises one or more abasic moieties. In some embodiments, the sense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, a sense strand comprises PS linkages between positions 1 and 2 relative to the 5′ end of the sense strand. In some embodiments, a sense strand comprises PS linkages between positions 1 and 2 relative to the 3′ end of the sense strand. In some embodiments, a sense strand comprises PS linkages between positions 1 and 2 relative to the 5′ end of the sense strand, and positions 1 and 2 relative to the 3′ end of the sense strand. In some embodiments, a sense strand comprises PS linkages between positions 1 and 2, and positions 2 and 3, relative to the 3′ end of the sense strand. In some embodiments, a sense strand comprises PS linkages between positions 1 and 2, and positions 2 and 3, relative to the 5′ end of the sense strand, and positions 1 and 2, and positions 2 and 3, relative to the 3′ end of the sense strand.

In some embodiments, the disclosure provides a double stranded nucleic acid comprising a sense strand; and an antisense strand having a 5′-end and a 3′-end, and consisting of modified nucleotides, wherein the modified nucleotides consist of 2′F-modified nucleotides located at positions 2, 4, 6, 8, 14, and 16 relative to the 5′-end of the antisense strand, and 2′O-Me modified nucleotides at the other positions of the antisense strand, and wherein the sense strand comprises 2′F-modified nucleotides located at and only at the following positions: (a) positions 9, 10, and 11; (b) positions 7, 9, and 11; (c) positions 7, 9, 10; or (d) positions 7, 10, and 11, and 2′O-Me modified nucleotides, 2′O—C₁₂₋₁₆ alkyl modified nucleotides or abasic moiety at the other positions of the sense strand; and wherein each of the sense and antisense strands comprise PS linkages at between positions 1 and 2, and positions 2 and 3, relative to their respective 5′ ends and 3′ ends.

In some embodiments, an antisense strand comprises 2′F-modified nucleotides at the following positions relative to the 5′ end of the antisense strand: 2, 4, 6, 8, 14, and 16, and does not comprise any 2′F-modified nucleotides other than those at the listed positions. In some embodiments, the antisense strand further comprises one or more additional modified nucleotides. In some embodiments, the antisense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the antisense strand comprises one or more abasic moieties. In some embodiments, the antisense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, an antisense strand comprises PS linkages between positions 1 and 2 relative to the 5′ end of the antisense strand. In some embodiments, an antisense strand comprises PS linkages between positions 1 and 2 relative to the 3′ end of the antisense strand. In some embodiments, an antisense strand comprises PS linkages between positions 1 and 2 relative to the 5′ end of the antisense strand, and positions 1 and 2 relative to the 3′ end of the antisense strand. In some embodiments, an antisense strand comprises PS linkages between positions 1 and 2, and positions 2 and 3, relative to the 3′ end of the antisense strand. In some embodiments, an antisense strand comprises PS linkages between positions 1 and 2, and positions 2 and 3, relative to the 5′ end of the antisense strand, and positions 1 and 2, and positions 2 and 3, relative to the 3′ end of the antisense strand.

In some embodiments, the antisense strand is hybridized to a sense strand. In some embodiments, the sense strand comprises one or more 2′F-modified nucleotides. In some embodiments, the sense strand comprises 2′F-modified nucleotides at the following positions: (a) positions 9, 10, and 11; (b) positions 7, 9, and 11; (c) positions 7, 9, 10; or (d) positions 7, 10, and 11. In some embodiments, the sense strand does not comprise any 2′F-modified nucleotides at positions other than the listed positions. In some embodiments, the sense strand further comprises one or more additional modified nucleotides. In some embodiments, the sense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the sense strand comprises one or more 2′O—C₁₂₋₁₆ alkyl modified nucleotides. In some embodiments, the sense strand comprises one or more abasic moieties. In some embodiments, the sense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, a sense strand comprises PS linkages between positions 1 and 2 relative to the 5′ end of the sense strand. In some embodiments, a sense strand comprises PS linkages between positions 1 and 2 relative to the 3′ end of the sense strand. In some embodiments, a sense strand comprises PS linkages between positions 1 and 2 relative to the 5′ end of the sense strand, and positions 1 and 2 relative to the 3′ end of the sense strand. In some embodiments, a sense strand comprises PS linkages between positions 1 and 2, and positions 2 and 3, relative to the 3′ end of the sense strand. In some embodiments, a sense strand comprises PS linkages between positions 1 and 2, and positions 2 and 3, relative to the 5′ end of the sense strand, and positions 1 and 2, and positions 2 and 3, relative to the 3′ end of the sense strand.

In some embodiments, the disclosure provides a double stranded nucleic acid comprising a sense strand; and an antisense strand having a 5′-end and a 3′-end, and consisting of modified nucleotides, wherein the modified nucleotides consist of 2′F-modified nucleotides located at positions 2, 6, 8, 14, and 16 relative to the 5′-end of the antisense strand, and 2′O-Me modified nucleotides at the other positions of the antisense strand, and wherein the sense strand comprises 2′F-modified nucleotides located at and only at the following positions: (a) positions 9, 10, and 11; (b) positions 7, 9, and 11; (c) positions 7, 9, 10; or (d) positions 7, 10, and 11, and 2′O-Me modified nucleotides, 2′O—C₁₂₋₁₆ alkyl modified nucleotides, or abasic moiety at the other positions of the sense strand; and wherein each of the sense and antisense strands comprise PS linkages at between positions 1 and 2, and positions 2 and 3, relative to their respective 5′ ends and 3′ ends.

In some embodiments, an antisense strand comprises 2′F-modified nucleotides at the following positions relative to the 5′ end of the antisense strand: 2, 6, 8, 14, and 16, and does not comprise any 2′F-modified nucleotides other than those at the listed positions. In some embodiments, the antisense strand further comprises one or more additional modified nucleotides. In some embodiments, the antisense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the antisense strand comprises one or more abasic moieties. In some embodiments, the antisense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, an antisense strand comprises PS linkages between positions 1 and 2 relative to the 5′ end of the antisense strand. In some embodiments, an antisense strand comprises PS linkages between positions 1 and 2 relative to the 3′ end of the antisense strand. In some embodiments, an antisense strand comprises PS linkages between positions 1 and 2 relative to the 5′ end of the antisense strand, and positions 1 and 2 relative to the 3′ end of the antisense strand. In some embodiments, an antisense strand comprises PS linkages between positions 1 and 2, and positions 2 and 3, relative to the 3′ end of the antisense strand. In some embodiments, an antisense strand comprises PS linkages between positions 1 and 2, and positions 2 and 3, relative to the 5′ end of the antisense strand, and positions 1 and 2, and positions 2 and 3, relative to the 3′ end of the antisense strand.

In some embodiments, the antisense strand is hybridized to a sense strand. In some embodiments, the sense strand comprises one or more 2′F-modified nucleotides. In some embodiments, the sense strand comprises 2′F-modified nucleotides at the following positions: (a) positions 9, 10, and 11; (b) positions 7, 9, and 11; (c) positions 7, 9, 10; or (d) positions 7, 10, and 11. In some embodiments, the sense strand does not comprise any 2′F-modified nucleotides at positions other than the listed positions. In some embodiments, the sense strand further comprises one or more additional modified nucleotides. In some embodiments, the sense strand comprises 1, 2, 3, 4, 5, 6, 7, 8, 9, 10, 11, 12, 13, 14, 15, 16, 17, 18, 19, 20, 21, 22, or 23 additional modified nucleotides. In some embodiments, the one or more additional modified nucleotides each comprise a 2′O-alkyl modification. In some embodiments, each of the one or more additional modified nucleotides comprises a 2′O-methyl (2′O-Me) modification. In some embodiments, the sense strand comprises one or more 2′O—C₁₂₋₁₆ alkyl modified nucleotides. In some embodiments, the sense strand comprises one or more abasic moieties. In some embodiments, the sense strand comprises 1 or more (e.g., 1, 2, 3, 4, 5, 6, 7, 8, or more) modified internucleotide linkages. In some embodiments, the modified internucleotide linkages are phosphorothioate (PS) linkages. In some embodiments, a sense strand comprises PS linkages between positions 1 and 2 relative to the 5′ end of the sense strand. In some embodiments, a sense strand comprises PS linkages between positions 1 and 2 relative to the 3′ end of the sense strand. In some embodiments, a sense strand comprises PS linkages between positions 1 and 2 relative to the 5′ end of the sense strand, and positions 1 and 2 relative to the 3′ end of the sense strand. In some embodiments, a sense strand comprises PS linkages between positions 1 and 2, and positions 2 and 3, relative to the 3′ end of the sense strand. In some embodiments, a sense strand comprises PS linkages between positions 1 and 2, and positions 2 and 3, relative to the 5′ end of the sense strand, and positions 1 and 2, and positions 2 and 3, relative to the 3′ end of the sense strand.

In some embodiments, a sense strand comprises a nucleotide sequence that is about 70%, 75%, 80%, 85%, 90%, 95%, or 99% identical to the sequence set forth in any one of SEQ ID NOs: 1-199. In some embodiments, the sense strand comprises a nucleotide sequence that is 100% identical to any one of SEQ ID NOs: 1-199. In some embodiments, the antisense strand comprises a nucleotide sequence that is about 70%, 75%, 80%, 85%, 90%, 95%, or 99% identical to any of the sequences set forth in any one of SEQ ID NOs: 1-199. In some embodiments, the antisense strand comprises a nucleotide sequence that is 100% identical to any one of SEQ ID NOs: 1-199.

In some embodiments, a double stranded nucleic acid comprises a sense strand and antisense strand having about 70%, 75%, 80%, 85%, 90%, 95%, or 99% sequence identity to the sense and antisense strand pairs set forth in any of SEQ ID NOs: 1-199. In some embodiments, the double stranded nucleic acid comprises a sense strand and antisense strand that is 100% identical to the sense and antisense strand pairs set forth in any of SEQ ID NOs: 1-199. In some embodiments, a double stranded nucleic acid comprises a sense strand having a modification pattern set forth in any one of SEQ ID NOs: 1-199. In some embodiments, a double stranded nucleic acid comprises an antisense strand having a modification pattern set forth in any one of SEQ ID NOs: 1-199.

Aspects of the disclosure relate to double stranded nucleic acids that are not conjugated to any other molecule(s) (e.g., delivery molecules, such as antibodies, lipids, sugars, etc.). In some embodiments, a double stranded nucleic acid is not conjugated to a lipid, for example cholesterol, tocopherol, etc. In some embodiments, a double stranded nucleic acid is not conjugated to an antibody, such as a transferrin receptor antibody, low density lipoprotein receptor (LDLR) antibody, etc. In some embodiments, a double stranded nucleic acid is not conjugated to a sugar, such as N-acetylgalactosamine (GalNAc). However, the skilled artisan recognizes that, in some embodiments, double stranded nucleic acids described herein may be attached to one or more delivery molecules to form a conjugate, as described in further detail below.

### Conjugates

Aspects of the disclosure relate to compositions comprising a double stranded nucleic acid as described herein optionally attached to a delivery molecule. As used herein, a “delivery molecule” refers to an agent that assists in delivery of the double stranded nucleic acid into a target cell, directing the tissue or cell specificity of the double stranded nucleic acid, and/or adds additional functionality to the double stranded nucleic acid. In some embodiments, a delivery molecule comprises a lipid, peptide, protein, antibody, or small molecule. Examples of delivery molecules include but are not limited to lipid (e.g., fatty acid, cholesterol, tocopherol, etc.), antibodies (e.g., cell receptor-specific antibodies, therapeutic antibodies, etc.), antigen binding fragments (e.g., Fab, single chain variable fragments (scFvs)), radioligands, sugars (e.g., GalNAc, etc.). In some embodiments, a double stranded nucleic acid is conjugated to a lipid (e.g., cholesterol, tocopherol). In some embodiments, a double stranded nucleic acid is conjugated to a peptide or antibody (e.g., protamine, cell receptor-specific antibodies, etc.). In some embodiments, the antibody is not a transferrin receptor antibody. In some embodiments, a double stranded nucleic acid is conjugated to a sugar (e.g., GalNAc).

In some embodiments, a delivery molecule is connected (e.g., conjugated, or bound) to a double stranded nucleic acid. The delivery molecule may be directly connected (e.g., by forming one or more interactions or bonds with a nucleotide or internucleotide linkage of the double stranded nucleic acid), or indirectly connected (e.g., via one or more linker molecules) to the double stranded nucleic acid. A delivery molecule may be connected (e.g., conjugated or bound) to a double stranded nucleic acid at any suitable position. For example, a delivery molecule may be conjugated to a terminus (e.g., a 5′ end or 3′ end) of a double stranded nucleic acid, or to an internal nucleotide or internucleotide linkage of a double stranded nucleic acid.

In some embodiments, a delivery molecule is directly conjugated to a double stranded nucleic acid. In some embodiments, a delivery molecule is indirectly conjugated to a double stranded nucleic acid, e.g., via a linker. In some embodiments, the linker comprises an amino acid linker (e.g., a glycine-rich linker, glycine-serine linker, proline linker, etc.), or a small molecule (e.g., poly-ethylene glycol linker, etc.). In some embodiments, the linker is a Mal-Tet-TCO linker, SMCC linker, or GDM linker and the delivery molecule is an antibody that does not bind to a transferrin receptor. In some embodiments, the linker is not a Mal-Tet-TCO linker, SMCC linker, or GDM linker and the antibody is an antibody that does not bind to a transferrin receptor. In some embodiments, the linker is a triethylene glycol (TEG) linker. In some embodiments, a conjugate comprises a double stranded nucleic acid comprising the sequence set forth in any one of SEQ ID NOs: 1-199, a TEG linker, and a cholesterol or tocopherol.

Aspects of the disclosure relate to conjugates comprising a double stranded nucleic acid as described herein and a GalNAc delivery molecule. Delivery molecules comprising N-acetylgalactosamine (GalNAc) are known to target the asialoglycoprotein receptor on liver cells and are one modality for delivery of double stranded nucleic acids to a desired tissue. In some embodiments, a double stranded nucleic acid is conjugated to a GalNAc delivery molecule comprising the structure of Formula II:

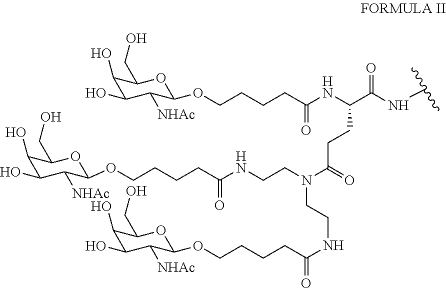 [SMILES: *NC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O]

In some embodiments, the double stranded nucleic acid is conjugated to Formula II via a linker. Suitable linkers are known in the art. In one embodiment, the linker comprises an alkyl chain, suitably C1-10 (e.g., a C6 chain). In some embodiments, the linker comprises a C6 amide (C6-NH₂). In a further embodiment, the linker is shown below as Linker 1 with connection points A and B (Formula III). In another embodiment the linker comprises a piperidine. In a further suitable embodiment, the linker is shown below as Linker 2 with connection points C and D (Formula IV).

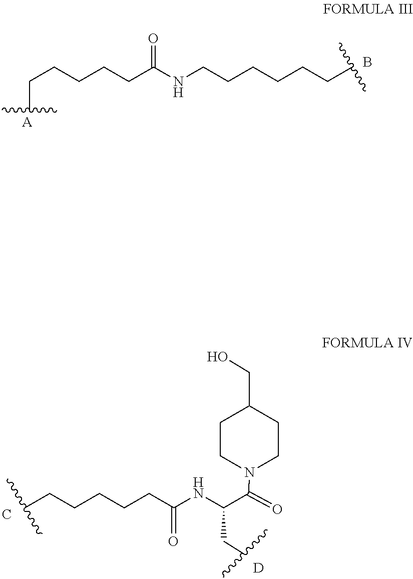

In an embodiment Linker 1 (Formula III), connection point A, or Linker 2 (Formula IV), connection point C, is conjugated to Formula II. In an embodiment, connection point A of Linker 1 is conjugated to Formula II and connection point B of Linker 1 is conjugated to a double stranded nucleic acid. In an embodiment, connection point C of Linker 2 is conjugated to Formula II and connection point D of Linker 2 is conjugated to a double stranded nucleic acid. In an embodiment, connection point A of Linker 1 is conjugated to Formula II and connection point B of Linker 1 is conjugated to a phosphate group which is conjugated to a double stranded nucleic acid. In an embodiment, connection point C of Linker 2 is conjugated to Formula II and connection point D of Linker 2 is conjugated to a phosphate group which is conjugated to a double stranded nucleic acid.

One of skill in the art will recognize that the linker may be on the 5′ or 3′ end of a double stranded nucleic acid, or attached to one of the internal nucleotide or nucleoside bases. One of skill in the art will also recognize that the linker may be linked or conjugated to the 5′ or 3′ end of a double stranded nucleic acid. One of skill in the art will also recognize that placement of a delivery molecule, such as the delivery molecules comprising Formula II, whether via a linker or not, on the 5′ end a double stranded nucleic acid may need to overcome potential inefficient loading of Ago2 loading, or other hindrance of the RISC complex activity. For example, for a delivery molecule comprising Formula II linked or directly conjugated to an siRNA comprising a sense and an antisense strand, placement of the delivery moiety at the 5′ end of the antisense strand may create difficulties for Ago2 loading and prevent efficient knockdown. In a suitable embodiment, the one or more double stranded nucleic acids comprise an siRNA comprising a sense and an antisense strand, and the delivery moiety comprising Formula II is present on the 3′ end of the sense strand. In a further embodiment, the delivery moiety comprising Formula II is conjugated to the 3′ end of the sense strand via a linker. In yet a further embodiment the linker comprises a ring structure, suitably a piperidine ring. In yet a further embodiment, the linker comprises Linker 2.

### Pharmaceutical Compositions

Compositions described herein may further comprise a pharmaceutical excipient, buffer, or diluent, and may be formulated for administration to a host cell ex vivo or in situ in an animal, and particularly a human being. Such compositions may further optionally comprise a liposome, a lipid, a lipid complex, a lipid nanoparticle (LNP), a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions related to the expression of a mutant protein, polypeptide or peptide or overexpression of a protein, polypeptide or peptide.

Formulations comprising pharmaceutically-acceptable excipients and/or carrier solutions are well-known to those of skill in the art, as is the development of suitable dosing and treatment regimens for using the particular compositions described herein in a variety of treatment regimens, including e.g., oral, parenteral, intravenous, intrathecal, subcutaneous, or intracisternal magna, intranasal, intra-articular, and intramuscular administration and formulation.

Typically, these formulations may contain at least about 0.1% of the therapeutic agent (e.g., nucleic acid or liposome comprising the nucleic acid) or more, although the percentage of the active ingredient(s) may, of course, be varied and may conveniently be between about 1 or 2% and about 70% or 90% or more of the weight or volume of the total formulation. The amount of therapeutic agent(s) in each therapeutically-useful composition may be prepared in such a way that a suitable dosage will be obtained in any given unit dose of the compound. Factors such as solubility, stability, bioavailability, biological half-life, route of administration, product shelf life, as well as other pharmacological considerations will be contemplated by one skilled in the art when preparing such pharmaceutical formulations. Additionally, a variety of dosages and treatment regimens may be desirable.

In certain circumstances, it will be desirable to deliver the nucleic acids or preparations thereof in suitably formulated pharmaceutical compositions disclosed herein; either subcutaneously, intraocularly, intravitreally, parenterally, intravenously, intracerebro-ventricularly, intracisternal magna, intramuscularly, intrathecally, orally, intraperitoneally, or by oral or nasal inhalation, or by direct injection to one or more cells, tissues, or organs.

The pharmaceutical formulations of the compositions suitable for injectable use include sterile aqueous solutions or dispersions. In some embodiments, the formulation is sterile and fluid to the extent that easy syringability exists. In some embodiments, the form is stable under the conditions of manufacture and storage, and is preserved against the contaminating action of microorganisms, such as bacteria and fungi. The carrier may be a solvent or dispersion medium containing, for example, water, saline, ethanol, polyol (e.g., glycerol, propylene glycol, and liquid polyethylene glycol, and the like), suitable mixtures thereof, vegetable oils or other pharmaceutically acceptable carriers such as those that are Generally Recognized as Safe (GRAS) by the United States Food and Drug Administration. Proper fluidity may be maintained, for example, by the use of a coating, such as lecithin, by the maintenance of the required particle size in the case of dispersion and by the use of surfactants. The nucleic acids may thus be delivered along with various other pharmaceutically acceptable agents as required in the particular instance. Such compositions may be purified from host cells or other biological sources, or alternatively may be chemically synthesized as described herein.

The amount of nucleic acids and/or nucleic acid compositions and time of administration of such compositions will be within the purview of the skilled artisan having the benefit of the present teachings. In some embodiments, the administration is a single administration. In some embodiments, the administration is more than one administration. In some circumstances, the administration schedule may be determined by the medical practitioner overseeing the administration of such compositions.

Toxicity and efficacy of the compositions utilized in methods of the present disclosure may be determined by standard pharmaceutical procedures, using either cells in culture or experimental animals to determine the LD50 (the dose lethal to 50% of the population) and/or IC50 (half maximal inhibitory concentration). The dose of the composition used for a therapeutic purpose may be chosen based on the ratio between toxicity and efficacy the therapeutic index and thus may be expressed as the ratio LD50/ED50 or LD50/IC50 (where “ED50” means the dose which is effective in 50% of the population and “IC50” means the dose which is effective for 50% inhibition of target mRNA translation). Those compositions that exhibit large therapeutic indices are preferred. While compositions that exhibit toxic side effects may be used, care should be taken to design a delivery system that minimizes the potential damage of such side effects. The dosage of compositions as described herein lies generally within a range that includes an ED50 or IC50 with little or no toxicity. The dosage may vary within this range depending upon the dosage form employed and the route of administration utilized.

Other aspects of the present disclosure relate to methods and preparations for use with a subject, such as human or non-human subjects, a host cell in situ in a subject, a host cell ex vivo, or a host cell derived from a subject. Non-limiting examples of subjects include dogs and cats; livestock such as horses, cattle, pigs, sheep, goats, and chickens; and other animals such as mice, rats, guinea pigs, and hamsters. In some embodiments, the subject is a human subject.

In some embodiments, one or more pharmaceutically acceptable excipients (including vehicles, carriers, diluents, and/or delivery polymers) are added to the pharmaceutical compositions including a therapeutic, thereby forming a pharmaceutical formulation suitable for in vivo delivery to a subject, such as a human.

A pharmaceutical composition or medicament includes a pharmacologically effective amount of at least one of the therapeutic and optionally one or more pharmaceutically acceptable excipients. Pharmaceutically acceptable excipients (excipients) are substances other than the Active Pharmaceutical Ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine).

The carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants such as preservatives, wetting agents, emulsifying agents, and dispersing agents. A carrier may also contain isotonic agents, such as sugars, polyalcohols, sodium chloride, and the like into the compositions.

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

### Methods

Aspects of the disclosure relate to methods for inhibiting or reducing a target mRNA in a cell, using a composition (e.g., a double stranded nucleic acid, conjugate, pharmaceutical composition, etc.) as described herein. In some embodiments, the methods comprise contacting a cell comprising the target mRNA with the double stranded nucleic acid (or a conjugate comprising the double stranded nucleic acid). In some embodiments, the methods comprise delivering the double stranded nucleic acid into a cell (e.g., contacting the cell with the double stranded nucleic acid), for example a mammalian cell or human cell. In some embodiments, the cell is in a subject (e.g., a human subject).

The amount of inhibition or reduction of target mRNA may vary. In some embodiments, the target mRNA is reduced between about 50% and about 100% relative to the amount of target mRNA prior to the administration. In some embodiments, the target mRNA is reduced by about 50%, 60%, 70%, 80%, 90%, 95%, 99%, or 100% relative to the amount of target mRNA prior to the administration. In some embodiments, the target mRNA is reduced between about 5-fold and about 100-fold relative to the amount of target mRNA prior to the administration. In some embodiments, the target mRNA is reduced by about 5-fold, 10-fold, 20-fold, 50-fold, or 100-fold relative to the amount of target mRNA prior to the administration. In some embodiments, the target mRNA is SNCA mRNA. In some embodiments, the target mRNA is APOE mRNA.

Aspects of the disclosure relate to double stranded nucleic acids having certain patterns of 2′-F modifications (e.g., having antisense strands having 2′F-modified nucleotides at positions 2, 5, 7, 14, and 16; 2, 3, 7, 14, and 16; 2, 5, 8, 14, and 16; 2, 4, 6, 8, 14, and 16; or 2, 6, 8, 14, and 16, relative to the 5′ end of the antisense strand) that have increased durability in vivo relative to double stranded nucleic acids having other modification patterns. In some embodiments, increased durability refers to the persistence of knock down or gene silencing mediated by the double stranded nucleic acid. In some embodiments, increased durability refers to resistance to degradation (e.g., enzymatic degradation) of the double stranded nucleic acid in vivo. In some embodiments, a double stranded nucleic acid as described herein persists for about 10% to about 100% longer in vivo relative to a double stranded nucleic acid having a different pattern of modifications (or an unmodified double stranded nucleic acid). In some embodiments, a double stranded nucleic acid as described herein persists for about 50%, 60%, 70%, 80%, 90%, 95%, 99%, or 100% longer in vivo than a double stranded nucleic acid having a different pattern of modifications (or an unmodified double stranded nucleic acid). In some embodiments, a double stranded nucleic acid as described herein persists for about 5-fold and about 100-fold longer in vivo relative to a double stranded nucleic acid having a different pattern of modifications (or an unmodified double stranded nucleic acid). In some embodiments, a double stranded nucleic acid as described herein persists for about 5-fold, 10-fold, 20-fold, 50-fold, or 100-fold longer in vivo relative to a double stranded nucleic acid having a different pattern of modifications (or an unmodified double stranded nucleic acid).

Aspects of the disclosure relate to double stranded nucleic acids having reduced (or decreased) off-target effects relative to double stranded nucleic acids having a different pattern of modifications (or an unmodified double stranded nucleic acid). In some embodiments, the off-targeting is reduced between about 50% and about 100% relative to a double stranded nucleic acid having a different pattern of modifications (or an unmodified double stranded nucleic acid). In some embodiments, the off-targeting is reduced by about 20%, 30%, 40%, 50%, 60%, 70%, 80%, 90%, 95%, 99%, or 100% relative to a double stranded nucleic acid having a different pattern of modifications (or an unmodified double stranded nucleic acid). In some embodiments, the off-targeting is reduced between about 2-fold and about 100-fold relative to a double stranded nucleic acid having a different pattern of modifications (or an unmodified double stranded nucleic acid). In some embodiments, the off-targeting is reduced by about 2-fold, 5-fold, 10-fold, 20-fold, 50-fold, or 100-fold relative to a double stranded nucleic acid having a different pattern of modifications (or an unmodified double stranded nucleic acid).

In some aspects, the disclosure provides a method for treating a subject having or suspected of having a disease, disorder, or condition. In some embodiments, a subject is a human (and may also be referred to as a “patient”). Treatment of a subject involves administration of a composition to the subject (e.g., double stranded nucleic acids, conjugates, pharmaceutical compositions, etc.) as described herein. As used herein, “treatment” or “treating” refers to all processes wherein there may be a slowing, controlling, delaying, or stopping of the development or progression of the disorders or disease disclosed herein, or ameliorating disorder or disease symptoms, but does not necessarily indicate a total elimination of all disorder or disease symptoms. Treatment includes administration of a composition (e.g., double stranded nucleic acids, conjugates, pharmaceutical compositions, etc.) described herein for treatment of a disease or condition in a patient, particularly in a human. In some embodiments, administration of the double stranded nucleic acids descried herein is a prophylactic treatment of a subject who does not and did not have a disease but is at risk of developing the disease or who did have a disease, and is not with a disease, but is at risk of regression of the disease. In certain embodiments, the subject is at a higher risk of developing a disease or at a higher risk of regression of the disease relative to an average healthy member of a population.

“Development” or “progression” of a disease, disorder, or condition means initial manifestations and/or ensuing progression of the disease, disorder, or condition. Development of the disease, disorder, or condition can be detectable and assessed using standard clinical techniques as well known in the art. However, development also refers to progression that may be undetectable. For purpose of this disclosure, development or progression refers to the biological course of the symptoms.

The optimal course of administration or delivery of the compositions (e.g., double stranded nucleic acids, conjugates, pharmaceutical compositions, etc.) of the disclosure may vary depending upon the desired result and/or on the subject to be treated. As used herein “administration” refers to contacting cells with double stranded nucleic acids and preparations thereof and can be performed in vitro (including ex vivo) or in vivo. Compositions (e.g., double stranded nucleic acids, conjugates, pharmaceutical compositions, etc.) provided herein can be administered a number of routes including, but not limited to, by oral administration, intravenous administration (e.g., systemic intravenous injection/administration), intrathecal administration, subcutaneous administration, or intracisternal magna administration to the brain.

In general, the most appropriate route of administration will depend upon a variety of factors including the nature of the agent (e.g., its stability in the environment of the patient), and/or the condition of the subject (e.g., whether the subject is able to tolerate oral administration, injection, etc.). In some embodiments, compositions are administered to a subject through only one administration route. In some embodiments, multiple administration routes may be exploited (e.g., serially, or simultaneously) for administration of the composition to a subject.

In some embodiments, an effective amount (e.g., an amount sufficient to decrease expression or activity of a target mRNA) is administered to a subject. An “effective amount” refers to an amount necessary (for periods of time and for the means of administration) to achieve the desired therapeutic result. An effective amount may vary according to factors such as the disease state, age, sex, and weight of the individual, and the ability of the double stranded nucleic acid to elicit a desired response in the individual. An effective amount is also one in which any toxic or detrimental effects of administration are outweighed by the therapeutically beneficial effects. The effective amount will depend primarily on factors such as the species, age, weight, health of the subject, and the tissue to be targeted, and may thus vary among animal and tissue. In some embodiments, an effective amount can be a combination of an effective dosage, frequency, and duration for administration During the course of treatment, administration of a composition (e.g., double stranded nucleic acids, conjugates, pharmaceutical compositions, etc.) may be altered or adjusted accordingly. For example, expression of the target mRNA and/or the protein product encoded by the target mRNA may be monitored to inform methods of use of the composition. Expression information may be obtained, for example, through measuring changes in the levels of the protein or RNA products of the target mRNA.

### EXAMPLES

### Example 1: Synthesis of the RNAi Agent

Single strands (sense and antisense) of the RNA duplexes were synthesized on solid support via a MerMade™ 12 (LGC Biosearch Technologies). The sequences of the unmodified and modified sense and antisense strands were shown in Tables 1-4. The mRNA Target 1 (APOE) and mRNA Target 2 (SNCA) molecules are not encoded by the same gene. The oligonucleotides were synthesized via phosphoramidite chemistry. In some embodiments, a sense strand described in Table 2 or Table 4 comprises a delivery molecule, such as a lipid (e.g., the cholesterol ester, cholesteryl). In some embodiments, the delivery molecule (e.g., lipid) is attached to the sense strand via a linker, for example a TEG linker.

TABLE 1
Unmodified Nucleic Acid Sequences of
dsRNA Targeting APOE.

| | | | | | Start |
|---|---|---|---|---|---|
| | Sense | SEQ | Antisense | SEQ | Position |
| dsRNA | Strand | ID | Strand | ID | mRNA |
| No. | (5′ to 3′) | NO. | (5′ to 3′) | NO. | Target 1 |
| 1 | AGGUUCUGUGG | 1 | UCAACGCAGCCC | 5 | 74 |
| | GCUGCGUUGA | | ACAGAACCUUC | | |
| 2 | GGCUGCGUUGC | 2 | UAUGUGACCAGC | 6 | 84 |
| | UGGUCACAUA | | AACGCAGCCCA | | |
| 3 | GGCCUACAAAU | 3 | UCCAGUUCCGAU | 7 | 339 |
| | CGGAACUGGA | | UUGUAGGCCUU | | |
| 4 | AUGGACGAGAC | 4 | UUCCUUCAUGGU | 8 | 313 |
| | CAUGAAGGAA | | CUCGUCCAUCA | | |

TABLE 2
Modified Nucleic Acid Sequences of dsRNA Targeting APOE.

| dsRNA | Sense Strand | SEQ ID | Antisense Strand | SEQ |
|---|---|---|---|---|
| No. | (5′ to 3′) | NO. | (5′ to 3′) | ID NO. |
| 1 | mA*mG*mGmUmUmCfUmGfUfGf | 9 | mU*fC*mAmAmCfGmCmAmGmCmC | 43 |
| | GmGmCmUmGmCmGmUmU*mG* | | mCmAfCmAfGmAmAmCmCmU*mU* | |
| | mA | | mC | |
| 1 | mA*mG*mGmUmUmCfUmGfUfGf | 10 | mU*fC*mAmAfCmGmCmAmGmCmC | 44 |
| | GmGmCmUmGmCmGmUmU*mG* | | mCmAfCmAfGmAmAmCmCmU*mU* | |
| | mA | | mC | |
| 1 | mA*mG*mGmUmUmCfUmGfUfGf | 11 | mU*fC*mAfAmCmGmCmAmGmCmC | 45 |
| | GmGmCmUmGmCmGmUmU*mG* | | mCmAfCmAfGmAmAmCmCmU*mU* | |
| | mA | | mC | |
| 1 | mA*mG*mGmUmUmCfUmGfUfGf | 12 | mU*fC*mAmAmCmGfCmAmGmCmC | 46 |
| | GmGmCmUmGmCmGmUmU*mG* | | mCmAfCmAfGmAmAmCmCmU*mU* | |
| | mA | | mC | |
| 1 | mA*mG*mGmUmUmCfUmGfUfGf | 13 | mU*fC*fAmAmCmGfCmAmGmCmCm | 47 |
| | GmGmCmUmGmCmGmUmU*mG* | | CmAfCmAfGmAmAmCmCmU*mU*mC | |
| | mA | | | |
| 1 | mA*mG*mGmUmUmCfUmGfUfGf | 14 | mU*fC*mAmAfCmGfCmAmGmCmCm | 48 |
| | GmGmCmUmGmCmGmUmU*mG* | | CmAfCmAfGmAmAmCmCmU*mU*mC | |
| | mA | | | |
| 1 | mA*mG*mGmUmUmCmUmGfU | 15 | mU*fC*mAmAfCmGfCmAmGmCmC | 49 |
| | fGfGmGmCmUmGmCmGmU | | mCmAfCmAfGmAmAmCmC | |
| | mU*mG*mA | | mU*mU*mC | |
| 2 | mG*mG*mCmUmGmCfGmUfUfGfC | 16 | mU*fA*mUmGmUfGmAmCmCmAmG | 50 |
| | mUmGmGmUmCmAmCmA*mU*m | | mCmAfAmCfGmCmAmGmCmC*mC* | |
| | A | | mA | |
| 2 | mG*mG*mCmUmGmCfGmUfUfGfC | 17 | mU*fA*mUmGmUfGmAmCmCmAmG | 51 |
| | mUmGmGmUmCmAmCmA*mU*m | | mCmAfAmCfGmCmAmGmCmC*mC* | |
| | A | | mA | |
| 2 | mG*mG*mCmUmGmCfGmUfUfGfC | 18 | mU*fA*mUmGfUmGmAmCmCmAmG | 52 |
| | mUmGmGmUmCmAmCmA*mU*m | | mCmAfAmCfGmCmAmGmCmC*mC* | |
| | A | | mA | |
| 2 | mG*mG*mCmUmGmCfGmUfUfGfC | 19 | mU*fA*mUfGmUmGmAmCmCmAmG | 53 |
| | mUmGmGmUmCmAmCmA*mU*m | | mCmAfAmCfGmCmAmGmCmC*mC* | |
| | A | | mA | |
| 2 | mG*mG*mCmUmGmCfGmUfUfGfC | 20 | mU*fA*mUmGmUmGfAmCmCmAmG | 54 |
| | mUmGmGmUmCmAmCmA*mU*m | | mCmAfAmCfGmCmAmGmCmC*mC* | |
| | A | | mA | |
| 2 | mG*mG*mCmUmGmCfGmUfUfGfC | 21 | mU*fA*fUmGmUmGfAmCmCmAmGm | 55 |
| | mUmGmGmUmCmAmCmA*mU*m | | CmAfAmCfGmCmAmGmCmC*mC*mA | |
| | A | | | |
| 2 | mG*mG*mCmUmGmCfGmUfUfGfC | 22 | mU*fA*mUmGfUmGfAmCmCmAmGm | 56 |
| | mUmGmGmUmCmAmCmA*mU*m | | CmAfAmCfGmCmAmGmCmC*mC*mA | |
| | A | | | |
| 2 | mG*mG*mCmUmGmCfGmUfUfGfC | 23 | mU*fA*mUmGfUmGfAmCmCmAmGm | 57 |
| | mUmGmGmUmCmAmCmA*mU*m | | CmAfAmCfGmCmAmGmCmC*mC*mA | |
| | A | | | |
| 2 | mG*mG*mCmUmGmCfGmUfUfGfC | 24 | mU*fA*mUmGfUmGfAmCmCmAmGm | 58 |
| | mUmGmGmUmCmAmCmA*mU*m | | CmAfAmCfGmCmAmGmCmC*mC*mA | |
| | A | | | |
| 2 | mG*mG*mCmUmGmCmGmUfU | 25 | mU*fA*mUmGfUmGfAmCmCmAmG | 59 |
| | fGfCmUmGmGmUmCmAmC | | mCmAfAmCfGmCmAmGmC | |
| | mA*mU*mA | | mC*mC*mA | |
| 2 | mG*mG*mCmUmGmCmGmUfU | 26 | mU*fA*mUmGfUmGfAmCmCmAmG | 60 |
| | fGfCmUmGmGmUmCmAmC | | mCmAfAmCfGmCmAmGmC | |
| | mA*mU*mA | | mC*mC*mA | |
| 2 | mG*mG*mCmUmGmCfGmUfUfGfC | 27 | VPm5U*fA*mUmGmUfGmAmCmCmA | 61 |
| | mUmGmGmUmCmAmCmA*mU*m | | mGmCmAfAmCfGmCmAmGmCmC*m | |
| | A | | C*mA | |
| 2 | mG*mG*mCmUmGmCfGmUfUfGfC | 28 | VPm5U*fA*mUmGfUmGfAmCmCmA | 62 |
| | mUmGmGmUmCmAmCmA*mU*m | | mGmCmAfAmCfGmCmAmGmCmC*m | |
| | A | | C*mA | |
| 3 | mG*mG*mCmCmUmAfCmAfAfAfU | 29 | mU*fC*mCmAmGfUmUmCmCmGmA | 63 |
| | mCmGmGmAmAmCmUmG*mG*m | | mUmUfUmGfUmAmGmGmCmC*mU* | |
| | A | | mU | |
| 3 | mG*mG*mCmCmUmAfCmAfAfAfU | 30 | mU*fC*mCmAfGmUmUmCmCmGmA | 64 |
| | mCmGmGmAmAmCmUmG*mG*m | | mUmUfUmGfUmAmGmGmCmC*mU* | |
| | A | | mU | |
| 3 | mG*mG*mCmCmUmAfCmAfAfAfU | 31 | mU*fC*mCfAmGmUmUmCmCmGmA | 65 |
| | mCmGmGmAmAmCmUmG*mG*m | | mUmUfUmGfUmAmGmGmCmC*mU* | |
| | A | | mU | |
| 3 | mG*mG*mCmCmUmAfCmAfAfAfU | 32 | mU*fC*mCmAmGmUfUmCmCmGmA | 66 |
| | mCmGmGmAmAmCmUmG*mG*m | | mUmUfUmGfUmAmGmGmCmC*mU* | |
| | A | | mU | |
| 3 | mG*mG*mCmCmUmAfCmAfAfAfU | 33 | mU*fC*fCmAmGmUfUmCmCmGmAm | 67 |
| | mCmGmGmAmAmCmUmG*mG*m | | UmUfUmGfUmAmGmGmCmC*mU*m | |
| | A | | U | |
| 3 | mG*mG*mCmCmUmAfCmAfAfAfU | 34 | mU*fC*mCmAfGmUfUmCmCmGmAm | 68 |
| | mCmGmGmAmAmCmUmG*mG*m | | UmUfUmGfUmAmGmGmCmC*mU*m | |
| | AP | | U | |
| 3 | mG*mG*mCmCmUmAmCmAfA | 35 | mU*fC*mCmAfGmUfUmCmCmGmA | 69 |
| | fAfUmCmGmGmAmAmCmU | | mUmUfUmGfUmAmGmGmC | |
| | mG*mG*mA | | mC*mU*mU | |
| 4 | mA*mU*mGmGmAmCfGmAfGfAfC | 36 | mU*fU*mCmCmUfUmCmAmUmGmG | 70 |
| | mCmAmUmGmAmAmGmG*mA*m | | mUmCfUmCfGmUmCmCmAmU*mC* | |
| | A | | mA | |
| 4 | mA*mU*mGmGmAmCfGmAfGfAfC | 37 | mU*fU*mCmCfUmUmCmAmUmGmG | 71 |
| | mCmAmUmGmAmAmGmG*mA*m | | mUmCfUmCfGmUmCmCmAmU*mC* | |
| | A | | nA | |
| 4 | mA*mU*mGmGmAmCfGmAfGfAfC | 38 | mU*fU*mCfCmUmUmCmAmUmGmG | 72 |
| | mCmAmUmGmAmAmGmG*mA*m | | mUmCfUmCfGmUmCmCmAmU*mC* | |
| | A | | mA | |
| 4 | mA*mU*mGmGmAmCfGmAfGfAfC | 39 | mU*fU*mCmCmUmUfCmAmUmGmG | 73 |
| | mCmAmUmGmAmAmGmG*mA*m | | mUmCfUmCfGmUmCmCmAmU*mC* | |
| | A | | mA | |
| 4 | mA*mU*mGmGmAmCfGmAfGfAfC | 40 | mU*fU*fCmCmUmUfCmAmUmGmGm | 74 |
| | mCmAmUmGmAmAmGmG*mA*m | | UmCfUmCfGmUmCmCmAmU*mC*m | |
| | A | | A | |
| 4 | mA*mU*mGmGmAmCfGmAfGfAfC | 41 | mU*fU*mCmCfUmUfCmAmUmGmGm | 75 |
| | mCmAmUmGmAmAmGmG*mA*m | | UmCfUmCfGmUmCmCmAmU*mC*m | |
| | A | | A | |
| 4 | mA*mU*mGmGmAmCmGmAfG | 42 | mU*fU*mCmCfUmUfCmAmUmGmG | 76 |
| | fAfCmCmAmUmGmAmAmG | | mUmCfUmCfGmUmCmCmA | |
| | mG*mA*mA | | mU*mC*mA | |

Abbreviations - “m” indicates 2′-OMe; “f” indicates 2′-fluoro; “*” indicates a modified linkage (e.g., a phosphorothioate linkage); and “VP” indicates 5′-vinylphosphonate.

TABLE 3
Unmodified Nucleic Acid Sequences of dsRNA Targeting SNCA.

| | | | | | Start |
|---|---|---|---|---|---|
| | | | | | posi- |
| | | | | | tion |
| | | | | | of |
| | | | | | target |
| | | | | | region |
| | | | | | on |
| RNAi | | SEQ | Antisense | SEQ | mRNA |
| Agent | Sense Strand | ID | Strand | ID | Target |
| No. | (5′ to 3′) | NO | (5′ to 3′) | NO | 2 |
| 1 | CUGUACAAGUGCUCA | 77 | UGGAACUGAGCACUUG | 78 | 701 |
| | GUUCCA | | UACAGGA | | |
| 2 | CUGUACAAGUGCUCA | 145 | UGGAACTGAGCACUUG | 147 | |
| | GTUCCA | | UACAGGA | | |
| 3 | UGUACAAGUGCUCAG | 146 | UUGGAACUGAGCACUU | 148 | |
| | UUCCAA | | GUACAG | | |
| 4 | UGUACAAGUGCUCAG | 146 | UUGGACUGAGCACUUG | 149 | |
| | UUCCAA | | UACAGG | | |
| 5 | UGUACAAGUGCUCAG | 146 | UUGGAAUGAGCACUUG | 150 | |
| | UUCCAA | | UACAGG | | |
| 39 | UGUACAAGUGCUCAG | 146 | UUGGAACGAGCACUUG | 151 | |
| | UUCCAA | | UACAGG | | |

TABLE 4
Modified Nucleic Acid Sequences of dsRNA Targeting SNCA

| dsRNA | | SEQ ID | Antisense Strand | SEQ ID |
|---|---|---|---|---|
| No. | Sense Strand (5′ to 3′) | NO. | (5′ to 3′) | NO. |
| 6 | mC*mU*mGmUmAmCfAmAfG(Ab)fG | 79 | VPmU*fG*mGmAmAfCmUmGmA | 112 |
| | mCmUmCmAmGmUmUmC*mC*mA | | mGmCmAmCfUmUfGmUmAmCm | |
| | | | AmG*mG*mA | |
| 6 | mC*mU*mGmUmAmCfAmArG(Ab)rG | 80 | VPmU*fG*mGmAfAmCmUfGmA | 113 |
| | mCmUmCmAmGmUmUmC*mC*mA | | mGmCmAmCfUmUfGmUmAmCm | |
| | | | AmG*mG*mA | |
| 6 | mC*mU*mGmUmAmCfAmAfG(Ab)fG | 81 | VPmU*fG*mGfAmAfCfUmGfAmG | 114 |
| | mCmUmCmAmGmUmUmC*mC*mA | | mCmAmCfUmUfGmUmAmCmAm | |
| | | | G*mG*mA | |
| 7 | mC*mU*mGmUmAmCfAmAfGfUfGm | 82 | VPmU*fG*mGmAfAfCfUfGmAmG | 115 |
| | CmUmCmAmGmUmUmC*mC*mA | | mCmAmCfUmUfGmUmAmCmAm | |
| | | | G*mG*mA | |
| 7 | mC*mU*mGmUmAmCfAmAfGfUfGm | 83 | VPmU*fG*mGmAmAmCmUmGm | 116 |
| | CmUmCmAmGmUmUmC*mC*mA | | AmGmCmAmCfUmUfGmUmAmC | |
| | | | mAmG*mG*mA | |
| 7 | mC*mU*mGmUmAmCfAmAfGfUfGm | 84 | VPmU*fG*mGmAfAmCmUfGmA | 117 |
| | CmUmCmAmGmUmUmC*mC*mA | | mGmCmAmCfUmUfGmUmAmCm | |
| | | | AmG*mG*mA | |
| 7 | mC*mU*mGmUmAmCfAmAfGfUfGm | 85 | VPmU*fG*mGmAfAmCmUmGmA | 118 |
| | CmUmCmAmGmUmUmC*mC*mA | | mGmCmAmCfUmUfGmUmAmCm | |
| | | | AmG*mG*mA | |
| 7 | mC*mU*mGmUmAmCfAmAfGfUfGm | 86 | VPmU*fG*mGmAmAfCmUmGmA | 119 |
| | CmUmCmAmGmUmUmC*mC*mA | | mGmCmAmCfUmUfGmUmAmCm | |
| | | | AmG*mG*mA | |
| 7 | mC*mU*mGmUmAmCfAmAfGUfGm | 87 | VPmU*fG*mGmAmAfCmUmGmA | 120 |
| | CmUmCmAmGmUmUmC*mC*mA | | mGmCmAmCfUmUfGmUmAmCm | |
| | | | AmG*mG*mA | |
| 7 | mC*mU*mGmUmAmCAmAfGfUfGm | 88 | VPmU*fG*mGmAmAfCmUmGmA | 121 |
| | CmUmCmAmGmUmUmC*mC*mA | | mGmCmAmCfUmUfGmUmAmCm | |
| | | | AmG*mG*mA | |
| 7 | mC*mU*mGmUmAmCfAmAfGfUfGm | 89 | VPmU*fG*mGmAmAfCmUmGmA | 122 |
| | CmUmCmAmGmUmUmC*mC*mG | | mGmCmAmCfUmUfGmUmAmCm | |
| | | | AmG*mG*mA | |
| 7 | mC*mU*mGmUmAmCfAmAfGfUfGm | 90 | VPmU*fG*mGfAmAmCmUmGmA | 123 |
| | CmUmCmAmGmUmUmC*mC*mA | | mGmCmAmCfUmUfGmUmAmCm | |
| | | | AmG*mG*mA | |
| 7 | mC*mU*mGmUmAmCmAmAfGfUfG | 91 | VPmU*fG*mGmAmAfCmUmGmA | 124 |
| | mCmUmCmAmGmUmUmC*mC*mA | | mGmCmAmCfUmUfGmUmAmCm | |
| | | | AmG*mG*mA | |
| 7 | fC*mU*fGmUfAmCfAmAfGfUfGm | 92 | VPmU*fG*mGmAmAfCmUmGmA | 125 |
| | CfUmCfAmGfUmUfC*mC*fA | | mGmCmAmCfUmUfGmUmAmCm | |
| | | | AmG*mG*mA | |
| 7 | mC*mU*mGmUmAmCfAmAfGfUfGm | 93 | VPmU*fG*mGfAmAfCmUfGmAm | 126 |
| | CmUmCmAmGmUmUmC*mC*mA | | GmCmAmCfUmUfGmUmAmCmA | |
| | | | mG*mG*mA | |
| 7 | mC*mU*mGmUmAmCfAmAfGfUfGm | 94 | VPmU*fG*mGmAmAfCmUfGmA | 127 |
| | CmUmCmAmGmUmUmC*mC*mA | | mGmCmAmCfUmUfGmUmAmCm | |
| | | | AmG*mG*mA | |
| 7 | mC*mU*mGmUmAmCfAmAfGfUfGm | 95 | VPmU*fG*mGmAmAfCmUmGmA | 128 |
| | CmUmCmAmGmTmUmC*mC*mA | | mGmCmAmCfUmUfGmUmAmCm | |
| | | | AmG*mG*mA | |
| 7 | mC*mU*mGmUmAmCfAmAfGfUfGm | 96 | VPmU*fG*mGfAmAfCmUfGmAfG | 129 |
| | CmUmCmAmGmUmUmC*mC*mA | | mCmAmCfUmUfGmUfAmCfAmG* | |
| | | | mG*mA | |
| 7 | mC*mU*mGmUmAmCrAmAfGrUfG | 97 | VPmU*fG*mGmAfAmCmUfGmA | 130 |
| | mCmUmCmAmGmUmUmC*mC*mA | | mGmCmAmCfUmUfGmUmAmCm | |
| | | | AmG*mG*mA | |
| 7 | mC*mU*mGmUmAmCmAmAfG(rU)f | 98 | VPmU*fG*mGmAfAmCmUfGmA | 131 |
| | GmCmUmCmAmGmUmUmC*mC*m | | mGmCmAmCfUmUfGmUmAmCm | |
| | A | | AmG*mG*mA | |
| 7 | mC*mU*mGmUmAmCfAmAfGfUrGm | 99 | VPmU*fG*mGmAfAmCmUfGmA | 132 |
| | CmUmCmAmGmUmUmC*mC*mA | | mGmCmAmCfUmUfGmUmAmCm | |
| | | | AmG*mG*mA | |
| 8 | mA*mC*fAmAfGfUfGmCmUmCmAm | 100 | VPmU*fG*mGmAmAfCmUmGmA | 133 |
| | GmUmUmC*mC*mA | | mGmCmAmCfUmUfGmUmAmCm | |
| | | | AmG*mG*mA | |
| 9 | mC*mG*mGmUmAmCfAmAfGfUfGm | 101 | VPmU*fG*mGmAmAfCmUmGmA | 134 |
| | CmUmCmAmGmUmUmC*mC*mA | | mGmCmAmCfUmUfGmUmAmCm | |
| | | | AmU*mG*mA | |
| 10 | mC*mU*mGmUmAmCfAmA(Ab)fUfG | 102 | VPmU*fG*mGmAfAmCmUfGmA | 135 |
| | mCmUmCmAmGmUmUmC*mC*mA | | mGmCmAmCfUmUfGmUmAmCm | |
| | | | AmG*mG*mA | |
| 11 | mC*mU*mGmUmAmCfAmAfGfU(Ab) | 103 | VPmU*fG*mGmAfAmCmUfGmA | 136 |
| | mCmUmCmAmGmUmUmC*mC*mA | | mGmCmAmCfUmUfGmUmAmCm | |
| | | | AmG*mG*mA | |
| 12 | mC*mU*mGmUmAmCfAmAfG(Ab)fG | 104 | VPmU*fG*mGmAmAmCmUmGm | 137 |
| | mCmUmCmAmGmUmUmC*mC*mA | | AmGmCmAmCfUmUfGmUmAmC | |
| | | | mAmG*mG*mA | |
| 12 | mC*mU*mGmUmAmCfAmAfG(Ab)fG | 105 | VPmU*fG*mGmAfAmCfUmGmA | 138 |
| | mCmUmCmAmGmUmUmC*mC*mA | | mGmCmAmCfUmUfGmUmAmCm | |
| | | | AmG*mG*mA | |
| 12 | mC*mU*mGmUmAmCfAmAfG(Ab)fG | 106 | VPmU*fG*mGmAfAmCmUfGmA | 139 |
| | mCmUmCmAmGmUmUmC*mC*mA | | mGmCmAmCfUmUfGmUmAmCm | |
| | | | AmG*mG*mA | |
| 12 | mC*mU*mGmUmAmCfAmAfG(Ab)fG | 107 | VPmU*fG*fGmAmAmCfUmGmA | 140 |
| | mCmUmCmAmGmUmUmC*mC*mA | | mGmCmAmCfUmUfGmUmAmCm | |
| | | | AmG*mG*mA | |
| 12 | mC*mU*mGmUmAmCmAmAfG(Ab)f | 108 | VPmU*fG*mGmAfAmCmUfGmA | 141 |
| | GmCmUmCmAmGmUmUmC*mC*m | | mGmCmAmCfUmUfGmUmAmCm | |
| | A | | AmG*mG*mA | |
| 12 | mC*mU*mGmUmAmCrAmAfG(Ab)fG | 109 | VPmU*fG*mGmAfAmCmUfGmA | 142 |
| | mCmUmCmAmGmUmUmC*mC*mA | | mGmCmAmCfUmUfGmUmAmCm | |
| | | | AmG*mG*mA | |
| 13 | mG*mU*mAmCrAmAfG(Ab)fGmCmU | 110 | VPmU*fG*mGmAfAmCmUfGmA | 143 |
| | mCmAmGmUmUmC*mC*mA | | mGmCmAmCfUmUfGmUmAmCm | |
| | | | A*mG*mG*mA | |
| 14 | mA*mC*rAmAfG(Ab)fGmCmUmCmA | 111 | VPmU*fG*mGmAfAmCmUfGmA | 144 |
| | mGmUmUmC*mC*mA | | mGmCmAmCfUmUfGmUmAmCm | |
| | | | A*mG*mG*mA | |
| 15 | mU*mG*mUmAmCmAfAmGfUfGfCm | 152 | VPmU*fU*mGmGmAfAmCmUmG | 176 |
| | UmCmAmGmUmUmCmC*mA*mA | | mAmGmCmAfCmUfUmGmUmAm | |
| | | | CmA*mG*mG | |
| 16 | mU*mG*mUmAmCmAfAmGfUfGfCm | 153 | VPmU*fU*mGmGmAfAmCmUmG | 177 |
| | UmCmAmGmUmUmCmC*mA*mA | | mAmGmCmAfCmUfUmGmUmAm | |
| | | | CmA*mG*mG | |
| 17 | mU*mG*mUmAmCmAmAmGfUfGfC | 154 | VPmU*fU*mGmGfAmAmCfUmG | 178 |
| | mUmCmAmGmUmUmCmC*mA*mA | | mAmGmCmAfCmUfUmGmUmAm | |
| | | | CmA*mG*mG | |
| 18 | mU*mG*mUmAmCmAmAmGfUfGfC | 155 | VPmU*fU*mGmGfAmAmCfUmG | 179 |
| | mUmCmAmGmUmUmCmC*mA*mA | | mAmGmCmAfCmUfUmGmUmAm | |
| | P | | CmA*mG*mG | |
| 19 | mU*mG*mUmAmCmAmAmGfUfGfC | 156 | VPmU*fU*mGmGfAmAmCfUmG | 180 |
| | mUmCmAmGmUmUmCmC*mA*mA | | mAmGmCmAfCmUfUmGmUmAm | |
| | | | CmA*mG*mG | |
| 20 | mU*mG*mUmAmCmAmAmGfUfGfC | 157 | VPmU*fU*mGmGfAmAmCfUmG | 181 |
| | mUmCmAmGmUmUmCmC*mA*mA | | mAmGmCmAfCmUfUmGmUmAm | |
| | | | C*mA*mG | |
| 21 | mU*mG*mUmAmCmAmAmGfUfGfC | 158 | VPmU*fU*mGmG(Ab)mAmCfUm | 182 |
| | mUmCmAmGmUmUmCmC*mA*mA | | GmAmGmCmAfCmUfUmGmUmA | |
| | | | mCmA*mG*mG | |
| 22 | mU*mG*mUmAmCmAmAmGfUfGfC | 159 | VPmU*fU*mGmGfA(Ab)mCfUmG | 183 |
| | mUmCmAmGmUmUmCmC*mA*mA | | mAmGmCmAfCmUfUmGmUmAm | |
| | | | CmA*mG*mG | |
| 23 | mU*mG*mUmAmCmAmAmGfUfGfC | 160 | VPmU*fU*mGmGfAmA(Ab)fUmG | 184 |
| | mUmCmAmGmUmUmCmC*mA*mA | | mAmGmCmAfCmUfUmGmUmAm | |
| | | | CmA*mG*mG | |
| 24 | mU*mG*mUmAmCmAmAmGfUfGfC | 161 | VPmU*fU*mGmGfAmAmC(Ab)m | 185 |
| | mUmCmAmGmUmUmCmC*mA*mA | | GmAmGmCmAfCmUfUmGmUmA | |
| | | | mCmA*mG*mG | |
| 25 | mU*mG*mUmAmCmAmAmGfUfGfC | 162 | VPmU*fU*mGmG(Ab)mAmCfUm | 186 |
| | mUmCmAmGmUmUmCmC*mA*mA | | GmAmGmCmAfCmUfUmGmUmA | |
| | | | mCmA*mG*mG | |
| 26 | mU*mG*mUmAmCmAmAmGfUfGfC | 163 | VPmU*fU*mGmGfA(Ab)mCfUmG | 187 |
| | mUmCmAmGmUmUmCmC*mA*mA | | mAmGmCmAfCmUfUmGmUmAm | |
| | | | CmA*mG*mG | |
| 27 | mU*mG*mUmAmCmAmAmGfUfGfC | 164 | VPmU*fU*mGmGfAmA(Ab)fUmG | 188 |
| | mUmCmAmGmUmUmCmC*mA*mA | | mAmGmCmAfCmUfUmGmUmAm | |
| | | | CmA*mG*mG | |
| 28 | mU*mG*mUmAmCmAmAmGfUfGfC | 165 | VPmU*fU*mGmGfAmAmC(Ab)m | 189 |
| | mUmCmAmGmUmUmCmC*mA*mA | | GmAmGmCmAfCmUfUmGmUmA | |
| | | | mCmA*mG*mG | |
| 29 | mC*mU*mGmUmAmCfAmAfGfUfGm | 166 | VPmU*fG*mGmAmAfCmUmGmA | 190 |
| | CmUmCmAmGmUmUmC*mC*mA | | mGmCmAmCfUmUfGmUmAmCm | |
| | | | AmG*mG*mA | |
| 30 | mC*mU*mGmUmAmCfAmAfGfUfGm | 167 | VPmU*fG*mGmAmAfCmUmGmA | 191 |
| | CmUmCmAmGmUmUmC*mC*mA | | mGmCmAmCfUmUfGmUmAmCm | |
| | | | AmG*mG*mA | |
| 31 | mC*mU*mGmUmAmCmAmAfGfUfG | 168 | VPmU*fG*mGmAfAmCmUfGmA | 192 |
| | mCmUmCmAmGmUmUmC*mC*mA | | mGmCmAmCfUmUfGmUmAmCm | |
| | | | AmG*mG*mA | |
| 32 | mC*mU*mGmUmAmCmAmAfGfUfG | 169 | VPmU*fG*mGmAfAmCfUmGmA | 193 |
| | mCmUmCmAmGmUmUmC*mC*mA | | mGmCmAmCfUmUfGmUmAmCm | |
| | | | AmG*mG*mA | |
| 33 | mC*mU*mGmUmAmCmAmAfGfUfG | 170 | VPmU*fG*mGmAfAmCmUfGmA | 194 |
| | mCmUmCmAmGmUmUmC*mC*mA | | mGmCmAmCfUmUfGmUmAmCm | |
| | | | AmG*mG*mA | |
| 34 | mC*mU*mGmUmAmCmAmAfGfUfG | 171 | VPmU*fG*mGmAfAmCmUfGmA | 195 |
| | mCmUmCmAmGmUmUmC*mC*mA | | mGmCmAmCfUmUfGmUmAmCm | |
| | | | AmG*mG*mA | |
| 35 | mC*mU*mGmUmAmCmAmAfGfUfG | 172 | VPmU*fG*mGmAmAmCmUmGm | 196 |
| | mCmUmCmAmGmUmUmC*mC*mA | | AmGmCmAmCfUmUfGmUmAmC | |
| | | | mAmG*mG*mA | |
| 36 | mC*mU*mGmUmAmCmAmAfGfUfG | 173 | VPmU*fG*mGdAfAmCdTfGmAm | 197 |
| | mCmUmCdAmGdTmUmC*mC*mA | | GmCmAmCfUmUfGmUmAmCmA | |
| | | | mG*mG*mA | |
| 37 | mC*mU*mGmUmAmCmAmAfGfUfG | 174 | VPmU*fG*mGmAfAmC*mUfGmA | 198 |
| | mCmUmCmAmGmUmUmC*mC*mA | | mGmCmAmCfUmUfGmUmAmCm | |
| | | | AmG*mG*mA | |
| 38 | mC*mU*mGmUmAmCmAmAfGfUfG | 175 | VPmU*fG*mGmAfA*mCmUfGmA | 199 |
| | mCmUmCmAmGmUmUmC*mC*mA | | mGmCmAmCfUmUfGmUmAmCm | |
| | | | AmG*mG*mA | |

Abbreviations-“m” indicates 2′-OMe; “f” indicates 2′-fluoro; “*” indicates a modified linkage (e.g., a phosphorothioate linkage); “VP” indicates 5′-vinylphosphonate; “(Ab)” indicates abasic moiety. “r” indicates a position comprising a ribose sugar; and “d” indicates a position comprising a deoxyribose sugar.

TABLE 5
Conjugates Comprising Modified Nucleic Acid Sequences of dsRNA Targeting
SNCA.

| dsRNA | | SEQ ID | | SEQ ID |
|---|---|---|---|---|
| No. | Sense Strand (5′ to 3′) | NO. | Antisense Strand (5′ to 3′) | NO. |
| 6 | mC*mU*mGmUmAmCfAmAfG(Ab)fG | 79 | VPmU*fG*mGmAmAfCmUmGmA | 112 |
| | mCmUmCmAmGmUmUmC*mC*mA[ | | mGmCmAmCfUmUfGmUmAmCm | |
| | TEG-Tocopherol] | | AmG*mG*mA | |
| 6 | mC*mU*mGmUmAmCfAmArG(Ab)rG | 80 | VPmU*fG*mGmAfAmCmUfGmA | 113 |
| | mCmUmCmAmGmUmUmC*mC*mA | | mGmCmAmCfUmUfGmUmAmCm | |
| | [TEG-Tocopherol] | | AmG*mG*mA | |
| 6 | mC*mU*mGmUmAmCfAmAfG(Ab)fG | 81 | VPmU*fG*mGfAmAfCfUmGfAmG | 114 |
| | mCmUmCmAmGmUmUmC*mC*mA | | mCmAmCfUmUfGmUmAmCmAm | |
| | [TEG-Tocopherol] | | G*mG*mA | |
| 7 | mC*mU*mGmUmAmCfAmAfGfUfGm | 82 | VPmU*fG*mGmAfAfCfUfGmAmG | 115 |
| | CmUmCmAmGmUmUmC*mC*mA[T | | mCmAmCfUmUfGmUmAmCmAm | |
| | EG-Tocopherol] | | G*mG*mA | |
| 7 | mC*mU*mGmUmAmCfAmAfGfUfGm | 83 | VPmU*fG*mGmAmAmCmUmGm | 116 |
| | CmUmCmAmGmUmUmC*mC*mA[T | | AmGmCmAmCfUmUfGmUmAmC | |
| | EG-Tocopherol] | | mAmG*mG*mA | |
| 7 | mC*mU*mGmUmAmCfAmAfGfUfGm | 84 | VPmU*fG*mGmAfAmCmUfGmA | 117 |
| | CmUmCmAmGmUmUmC*mC*mA[T | | mGmCmAmCfUmUfGmUmAmCm | |
| | EG-Tocopherol] | | AmG*mG*mA | |
| 7 | mC*mU*mGmUmAmCfAmAfGfUfGm | 85 | VPmU*fG*mGmAfAmCmUmGmA | 118 |
| | CmUmCmAmGmUmUmC*mC*mA[T | | mGmCmAmCfUmUfGmUmAmCm | |
| | EG-Tocopherol] | | AmG*mG*mA | |
| 7 | mC*mU*mGmUmAmCfAmAfGfUfGm | 86 | VPmU*fG*mGmAmAfCmUmGmA | 119 |
| | CmUmCmAmGmUmUmC*mC*mA[T | | mGmCmAmCfUmUfGmUmAmCm | |
| | EG-Tocopherol] | | AmG*mG*mA | |
| 7 | mC*mU*mGmUmAmCfAmAfGUfGm | 87 | VPmU*fG*mGmAmAfCmUmGmA | 120 |
| | CmUmCmAmGmUmUmC*mC*mA[T | | mGmCmAmCfUmUfGmUmAmCm | |
| | EG-Tocopherol] | | AmG*mG*mA | |
| 7 | mC*mU*mGmUmAmCAmAfGfUfGm | 88 | VPmU*fG*mGmAmAfCmUmGmA | 121 |
| | CmUmCmAmGmUmUmC*mC*mA[T | | mGmCmAmCfUmUfGmUmAmCm | |
| | EG-Tocopherol] | | AmG*mG*mA | |
| 7 | mC*mU*mGmUmAmCfAmAfGfUfGm | 89 | VPmU*fG*mGmAmAfCmUmGmA | 122 |
| | CmUmCmAmGmUmUmC*mC*mG[T | | mGmCmAmCfUmUfGmUmAmCm | |
| | EG-Tocopherol] | | AmG*mG*mA | |
| 7 | mC*mU*mGmUmAmCfAmAfGfUfGm | 90 | VPmU*fG*mGfAmAmCmUmGmA | 123 |
| | CmUmCmAmGmUmUmC*mC*mA[T | | mGmCmAmCfUmUfGmUmAmCm | |
| | EG-Tocopherol] | | AmG*mG*mA | |
| 7 | mC*mU*mGmUmAmCmAmAfGfUfG | 91 | VPmU*fG*mGmAmAfCmUmGmA | 124 |
| | mCmUmCmAmGmUmUmC*mC*mA[ | | mGmCmAmCfUmUfGmUmAmCm | |
| | TEG-Tocopherol] | | AmG*mG*mA | |
| 7 | fC*mU*fGmUfAmCfAmAfGfUfGmCfU | 92 | VPmU*fG*mGmAmAfCmUmGmA | 125 |
| | mCfAmGfUmUfC*mC*fA[TEG- | | mGmCmAmCfUmUfGmUmAmCm | |
| | Tocopherol] | | AmG*mG*mA | |
| 7 | mC*mU*mGmUmAmCfAmAfGfUfGm | 93 | VPmU*fG*mGfAmAfCmUfGmAm | 126 |
| | CmUmCmAmGmUmUmC*mC*mA[T | | GmCmAmCfUmUfGmUmAmCmA | |
| | EG-Tocopherol] | | mG*mG*mA | |
| 7 | mC*mU*mGmUmAmCfAmAfGfUfGm | 94 | VPmU*fG*mGmAmAfCmUfGmA | 127 |
| | CmUmCmAmGmUmUmC*mC*mA[T | | mGmCmAmCfUmUfGmUmAmCm | |
| | EG-Tocopherol] | | AmG*mG*mA | |
| 7 | mC*mU*mGmUmAmCfAmAfGfUfGm | 95 | VPmU*fG*mGmAmAfCmUmGmA | 128 |
| | CmUmCmAmGmTmUmC*mC*mA[TE | | mGmCmAmCfUmUfGmUmAmCm | |
| | G-Tocopherol] | | AmG*mG*mA | |
| 7 | mC*mU*mGmUmAmCfAmAfGfUfGm | 96 | VPmU*fG*mGfAmAfCmUfGmAfG | 129 |
| | CmUmCmAmGmUmUmC*mC*mA[T | | mCmAmCfUmUfGmUfAmCfAmG* | |
| | EG-Tocopherol] | | mG*mA | |
| 7 | mC*mU*mGmUmAmCrAmAfGrUfG | 97 | VPmU*fG*mGmAfAmCmUfGmA | 130 |
| | mCmUmCmAmGmUmUmC*mC*mA | | mGmCmAmCfUmUfGmUmAmCm | |
| | [TEG-Tocopherol] | | AmG*mG*mA | |
| 7 | mC*mU*mGmUmAmCmAmAfG(rU)f | 98 | VPmU*fG*mGmAfAmCmUfGmA | 131 |
| | GmCmUmCmAmGmUmUmC*mC*m | | mGmCmAmCfUmUfGmUmAmCm | |
| | A [TEG-Tocopherol] | | AmG*mG*mA | |
| 7 | mC*mU*mGmUmAmCfAmAfGfUrGm | 99 | VPmU*fG*mGmAfAmCmUfGmA | 132 |
| | CmUmCmAmGmUmUmC*mC*mA | | mGmCmAmCfUmUfGmUmAmCm | |
| | [TEG-Tocopherol] | | AmG*mG*mA | |
| 8 | mA*mC*fAmAfGfUfGmCmUmCmAm | 100 | VPmU*fG*mGmAmAfCmUmGmA | 133 |
| | GmUmUmC*mC*mA[TEG- | | mGmCmAmCfUmUfGmUmAmCm | |
| | Tocopherol] | | AmG*mG*mA | |
| 9 | mC*mG*mGmUmAmCfAmAfGfUfGm | 101 | VPmU*fG*mGmAmAfCmUmGmA | 134 |
| | CmUmCmAmGmUmUmC*mC*mA[T | | mGmCmAmCfUmUfGmUmAmCm | |
| | EG-Tocopherol] | | AmU*mG*mA | |
| 10 | mC*mU*mGmUmAmCfAmA(Ab)fUfG | 102 | VPmU*fG*mGmAfAmCmUfGmA | 135 |
| | mCmUmCmAmGmUmUmC*mC*mA | | mGmCmAmCfUmUfGmUmAmCm | |
| | [TEG-Tocopherol] | | AmG*mG*mA | |
| 11 | mC*mU*mGmUmAmCfAmAfGfU(Ab) | 103 | VPmU*fG*mGmAfAmCmUfGmA | 136 |
| | mCmUmCmAmGmUmUmC*mC*mA | | mGmCmAmCfUmUfGmUmAmCm | |
| | [TEG-Tocopherol] | | AmG*mG*mA | |
| 12 | mC*mU*mGmUmAmCfAmAfG(Ab)fG | 104 | VPmU*fG*mGmAmAmCmUmGm | 137 |
| | mCmUmCmAmGmUmUmC*mC*mA | | AmGmCmAmCfUmUfGmUmAmC | |
| | [TEG-Tocopherol] | | mAmG*mG*mA | |
| 12 | mC*mU*mGmUmAmCfAmAfG(Ab)fG | 105 | VPmU*fG*mGmAfAmCfUmGmA | 138 |
| | mCmUmCmAmGmUmUmC*mC*mA | | mGmCmAmCfUmUfGmUmAmCm | |
| | [TEG-Tocopherol] | | AmG*mG*mA | |
| 12 | mC*mU*mGmUmAmCfAmAfG(Ab)fG | 106 | VPmU*fG*mGmAfAmCmUfGmA | 139 |
| | mCmUmCmAmGmUmUmC*mC*mA | | mGmCmAmCfUmUfGmUmAmCm | |
| | [TEG-Tocopherol] | | AmG*mG*mA | |
| 12 | mC*mU*mGmUmAmCfAmAfG(Ab)fG | 107 | VPmU*fG*fGmAmAmCfUmGmA | 140 |
| | mCmUmCmAmGmUmUmC*mC*mA | | mGmCmAmCfUmUfGmUmAmCm | |
| | [TEG-Tocopherol] | | AmG*mG*mA | |
| 12 | mC*mU*mGmUmAmCmAmAfG(Ab)f | 108 | VPmU*fG*mGmAfAmCmUfGmA | 141 |
| | GmCmUmCmAmGmUmUmC*mC*m | | mGmCmAmCfUmUfGmUmAmCm | |
| | A [TEG-Tocopherol] | | AmG*mG*mA | |
| 12 | mC*mU*mGmUmAmCrAmAfG(Ab)fG | 109 | VPmU*fG*mGmAfAmCmUfGmA | 142 |
| | mCmUmCmAmGmUmUmC*mC*mA | | mGmCmAmCfUmUfGmUmAmCm | |
| | [TEG-Tocopherol] | | AmG*mG*mA | |
| 13 | mG*mU*mAmCrAmAfG(Ab)fGmCmU | 110 | VPmU*fG*mGmAfAmCmUfGmA | 143 |
| | mCmAmGmUmUmC*mC*mA[TEG- | | mGmCmAmCfUmUfGmUmAmCm | |
| | Tocopherol] | | A*mG*mG*mA | |
| 14 | mA*mC*rAmAfG(Ab)fGmCmUmCmA | 111 | VPmU*fG*mGmAfAmCmUfGmA | 144 |
| | mGmUmUmC*mC*mA[TEG- | | mGmCmAmCfUmUfGmUmAmCm | |
| | Tocopherol] | | A*mG*mG*mA | |
| 15 | mU*mG*mUmAmCmAfAmGfUfGfCm | 152 | VPmU*fU*mGmGmAfAmCmUmG | 176 |
| | UmCmAmGmUmUmCmC*mA*mA[T | | mAmGmCmAfCmUfUmGmUmAm | |
| | EG-Tocopherol] | | CmA*mG*mG | |
| 17 | mU*mG*mUmAmCmAmAmGfUfGfC | 154 | VPmU*fU*mGmGfAmAmCfUmG | 178 |
| | mUmCmAmGmUmUmCmC*mA*mA[ | | mAmGmCmAfCmUfUmGmUmAm | |
| | TEG-Cholesterol] | | CmA*mG*mG | |
| 18 | mU*mG*mUmAmCmAmAmGfUfGfC | 155 | VPmU*fU*mGmGfAmAmCfUmG | 179 |
| | mUmCmAmGmUmUmCmC*mA*mA | | mAmGmCmAfCmUfUmGmUmAm | |
| | P[TEG-Cholesterol] | | CmA*mG*mG | |
| 20 | mU*mG*mUmAmCmAmAmGfUfGfC | 157 | VPmU*fU*mGmGfAmAmCfUmGm | 181 |
| | mUmCmAmGmUmUmCmC*mA*mA[ | | AmGmCmAfCmUfUmGmUmAmC* | |
| | TEG-Tocopherol] | | mA*mG | |
| 25 | mU*mG*mUmAmCmAmAmGfUfGfC | 162 | VPmU*fU*mGmG(Ab)mAmCfUm | 186 |
| | mUmCmAmGmUmUmCmC*mA*mA[ | | GmAmGmCmAfCmUfUmGmUmA | |
| | TEG-Cholesterol] | | mCmA*mG*mG | |
| 26 | mU*mG*mUmAmCmAmAmGfUfGfC | 163 | VPmU*fU*mGmGfA(Ab)mCfUmG | 187 |
| | mUmCmAmGmUmUmCmC*mA*mA[ | | mAmGmCmAfCmUfUmGmUmAm | |
| | TEG-Cholesterol] | | CmA*mG*mG | |
| 27 | mU*mG*mUmAmCmAmAmGfUfGfC | 164 | VPmU*fU*mGmGfAmA(Ab)fUmG | 188 |
| | mUmCmAmGmUmUmCmC*mA*mA[ | | mAmGmCmAfCmUfUmGmUmAm | |
| | TEG-Cholesterol] | | CmA*mG*mG | |
| 28 | mU*mG*mUmAmCmAmAmGfUfGfC | 165 | VPmU*fU*mGmGfAmAmC(Ab)m | 189 |
| | mUmCmAmGmUmUmCmC*mA*mA[ | | GmAmGmCmAfCmUfUmGmUmA | |
| | TEG-Cholesterol] | | mCmA*mG*mG | |
| 30 | mC*mU*mGmUmAmCfAmAfGfUfGm | 167 | VPmU*fG*mGmAmAfCmUmGmA | 191 |
| | CmUmCmAmGmUmUmC*mC*mA[T | | mGmCmAmCfUmUfGmUmAmCm | |
| | EG-Cholesterol] | | AmG*mG*mA | |
| 31 | mC*mU*mGmUmAmCmAmAfGfUfG | 168 | VPmU*fG*mGmAfAmCmUfGmA | 192 |
| | mCmUmCmAmGmUmUmC*mC*mA[ | | mGmCmAmCfUmUfGmUmAmCm | |
| | TEG-Cholesterol] | | AmG*mG*mA | |
| 32 | mC*mU*mGmUmAmCmAmAfGfUfG | 169 | VPmU*fG*mGmAfAmCfUmGmA | 193 |
| | mCmUmCmAmGmUmUmC*mC*mA[ | | mGmCmAmCfUmUfGmUmAmCm | |
| | TEG-Cholesterol] | | AmG*mG*mA | |
| 34 | mC*mU*mGmUmAmCmAmAfGfUfG | 171 | VPmU*fG*mGmAfAmCmUfGmA | 195 |
| | mCmUmCmAmGmUmUmC*mC*mA[ | | mGmCmAmCfUmUfGmUmAmCm | |
| | TEG-Tocopherol] | | AmG*mG*mA | |
| 35 | mC*mU*mGmUmAmCmAmAfGfUfG | 172 | VPmU*fG*mGmAmAmCmUmGm | 196 |
| | mCmUmCmAmGmUmUmC*mC*mA[ | | AmGmCmAmCfUmUfGmUmAmC | |
| | TEG-Cholesterol] | | mAmG*mG*mA | |
| 36 | mC*mU*mGmUmAmCmAmAfGfUfG | 173 | VPmU*fG*mGdAfAmCdTfGmAm | 197 |
| | mCmUmCdAmGdTmUmC*mC*mA[T | | GmCmAmCfUmUfGmUmAmCmA | |
| | EG-Tocopherol] | | mG*mG*mA | |
| 37 | mC*mU*mGmUmAmCmAmAfGfUfG | 174 | VPmU*fG*mGmAfAmC*mUfGmA | 198 |
| | mCmUmCmAmGmUmUmC*mC*mA[ | | mGmCmAmCfUmUfGmUmAmCm | |
| | TEG-Tocopherol] | | AmG*mG*mA | |
| 38 | mC*mU*mGmUmAmCmAmAfGfUfG | 175 | VPmU*fG*mGmAfA*mCmUfGmA | 199 |
| | mCmUmCmAmGmUmUmC*mC*mA[ | | mGmCmAmCfUmUfGmUmAmCm | |
| | TEG-Tocopherol] | | AmG*mG*mA | |

Abbreviations-“m” indicates 2′-OMe; “f” indicates 2′-fluoro; “*” indicates a modified linkage (e.g., a phosphorothioate linkage); “VP” indicates 5′-vinylphosphonate; “(Ab)” indicates abasic moiety. “r” indicates a position comprising a ribose sugar; and “d” indicates a position comprising a deoxyribose sugar.

In some embodiments, a conjugate described herein comprises a sequence set forth in Table 2 or Table 4 or Table 5. In some embodiments, a conjugate described herein comprises a double-stranded RNA set forth in Table 2 or Table 4 or Table 5 (e.g., a conjugate comprising a sense strand and an antisense strand of a double-stranded RNA set forth in Table 2 or Table 4 or Table 5, wherein the sense-strand is conjugated to tocopherol or cholesterol). In some embodiments, a conjugate described herein comprises a sequence set forth in Table 2 or Table 4 or Table 5 (e.g., a sense strand) which is bonded (e.g., covalently bonded) to a linker described herein (e.g., a TEG linker, such as one which connects a sense strand of a double-stranded RNA to tocopherol or cholesterol).

For the sense strands, the types of solid supports were universal CPG: Universal UnyLinker (Chemgenes, Catalog No. AT273-27), 3′ Teg-Tocopherol (LGC Biosearch Technologies, Catalog No. BG7-1190), and 3′ Teg-Cholesterol (Chemgenes, Catalog No. N-9166-05) were purchased commercially. For all the antisense strands, commercially available standard support was utilized and dependent on sequence. Standard reagents were used in the oligo synthesis (Table 6), where 0.1M xanthane hydride in pyridine was used as the sulfurization reagent and 20% DEA in ACN was used as an auxiliary wash post synthesis. All monomers (Table 7) were made at 0.1M in ACN and contained a molecular sieves trap bag.

The oligonucleotides were cleaved and deprotected (C/D) at 45° C. for 20 hours. The sense strands were C/D from the CPG using ammonia hydroxide (28-30%, cold), whereas 3% DEA in ammonia hydroxide (28-30%, cold) was used for the antisense strands. C/D was determined complete by IP-RP LCMS when the resulting mass data confirmed the identity of sequence. Dependent on scale, the CPG was filtered via 0.45 um PVDF syringeless filter, 0.22 um PVDF Steriflip® vacuum filtration or 0.22 um PVDF Stericup® Quick release. The CPG was back washed/rinsed with either 30% ACN/RNAse free water or 30% EtOH/RNAse free water then filtered through the same filtering device and combined with the first filtrate. This was repeated twice. The material was then divided evenly into 50 mL falcon tubes to remove organics via Genevac™. After concentration, the crude oligonucleotides were diluted back to synthesized scale with RNAse free water and filtered either by 0.45 μm PVDF syringeless filter, 0.22 μm PVDF Steriflip® vacuum filtration or 0.22 μm PVDF Stericup® Quick release.

The crude oligonucleotides were purified via AKTA™ Pure purification system using either anion-exchange (AEX) or reverse phase (RP) a source 15Q-RP column. For AEX, an ES Industry Source™ 15Q column maintaining column temperature at 65° C. with MPA: 20 mM NaH₂PO₄, 15% ACN, pH 7.4 and MPB: 20 mM NaH₂PO₄, 1M NaBr, 15% ACN, pH 7.4. For RP, a Source™ 15Q-RP column with MPA: 50 mM NaOAc with 10% ACN and MPB: 50 mM NaOAc with 80% ACN. In all cases, fractions which contained a mass purity greater than 85% without impurities >5% where combined.

The purified oligonucleotides were desalted using 15 mL 3K MWCO centrifugal spin tubes at 3500×g for ~30 min. The oligonucleotides were rinsed with RNAse free water until the eluent conductivity reached <100 usemi/cm. After desalting was complete, 2-3 mL of RNAse free water was added then aspirated 10 times, the retainment was transferred to a 50 mL falcon tube, this was repeated until complete transfer of oligo by measuring concentration of compound on filter via nanodrop. The final oligonucleotide was then nano filtered 2× via 15 mL 100K MWCO centrifugal spin tubes at 3500×g for 2 min. The final desalted oligonucleotides were analyzed for concentration (nano drop at A260), characterized by IP-RP LCMS for mass purity and UPLC for UV-purity.

For the preparation of duplexes, equimolar amounts of sense and antisense strand were combined and heated at 65° C. for 10 minutes then slowly cooled to ambient temperature over 40 minutes. Integrity of the duplex was confirmed by UPLC analysis and characterized by LCMS using IP-RP. All duplexes were nano filtered then endotoxin levels measured via Charles River Endosafe® Cartridge Device to give the final compounds of conjugated RNAi. For in vivo analysis, the appropriate amount of duplex was lyophilized then reconstituted in 1×PBS for rodent studies and a CSF for non-human primate studies.

TABLE 6
Oligonucleotide Synthesis Reagents
Reagents

Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water,
70:20:10)
Deblock Solution, 3% TCA in DCM (w/v)
Acetonitrile (Anhydrosolv, Water max. 10 ppm)
Xanthane Hydride (0.1M in Pyridine)
Diethylamine (20% in Acetonitrile)

TABLE 7
Phosphoramidites

| Phosphoramidite | Abbreviation | Supplier | Catalog # | CAS |
|---|---|---|---|---|
| DMT-2′-F-A(Bz)-CE | fA | Hongene | PD1-001 | 136834-22-5 |
| Phosphoamidite | | | | |
| DMT-2′-F-C(Ac)-CE | fC | Hongene | PD3-001 | 159414-99-0 |
| Phosphoamidite | | | | |
| DMT-2′-F-G(iBu)-CE | fG | Hongene | PD2-002 | 144089-97-4 |
| Phosphoamidite | | | | |
| DMT-2′-F-U-CE | fU | Hongene | PD5-001 | 146954-75-8 |
| Phosphoamidite | | | | |
| DMT-2′-O-Me-A(Bz)- | mA | Hongene | PR1-001 | 110782-31-5 |
| CE Phosphoamidite | | | | |
| DMT-2′-O-Me-C(Ac)- | mC | Hongene | PR3-001 | 199593-09-4 |
| CE Phosphoamidite | | | | |
| DMT-2′-O-Me-G(iBu)- | mG | Hongene | PR2-002 | 150780-67-9 |
| CE Phosphoamidite | | | | |
| DMT-2′-O-Me-U-CE | mU | Hongene | PR5-001 | 110764-79-9 |
| Phosphoamidite | | | | |
| 5′bis(POM) vinyl | POM-VPmU | Hongene | PR5-032 | BVPMUP23B2A1 |
| phosphate-2′-Ome- | | | | |
| U3′CE | | | | |
| phosphoroamidite | | | | |
| Reverse Abasic | iAb | Chemgenes | ANP-1422 | 401813-16-9 |
| phosphoroamidite | | | | |
| Abasic | Ab | Chemgenes | ANP-7058 | 129821-76-7 |
| phosphoroamidite | | | | |
| 5′-O-Dimethoxytrityl- | rA | LGC | LK-2306 | 104992-55-4 |
| N6-benzoyl-2′-O- | | | | |
| TBDMSi-adenosine-3′- | | | | |
| O-(beta-cyanoethyl- | | | | |
| N,N-diisopropyl) | | | | |
| phosphoramidite | | | | |
| 5′-O-Dimethoxytrityl- | rU | LGC | LK-2040 | 118362-03-1 |
| 2′-O-TBDMSi-uridine- | | | | |
| 3′-O-(beta- | | | | |
| cyanoethyl-N,N- | | | | |
| diisopropyl) | | | | |
| phosphoramidite | | | | |
| 5′-O-Dimethoxytrityl- | rG | Thermo | XI2142 | 147201-04-5 |
| N2-isobutyryl-2′-O- | | Fisher | | |
| TBDMSi-guanosine-3′- | | Scientific | | |
| O-(Beta-cyanoethyl- | | | | |
| N,N-diisopropyl) | | | | |
| phosphoramidite | | | | |

### Example 2. In Vitro Characterization of the RNAi Agents

Selected RNAi agents were tested for mRNA Target 1 (APOE) or mRNA Target 2 (SNCA) inhibition in cultured cells, including HEP3B, SH-SY5Y, iPSC astrocyte cells or mouse primary cortical neurons.

### Materials and Methods

HEP3B, SH-SY5Y and Astrocyte Cell Culture and RNAi Treatment and Analysis: SH-SY5Y cells (ATCC CRL-2266) were derived from the SK-N-SH neuroblastoma cell line (Ross, R. A., et al., 1983. J Natl Cancer Inst 71, 741-747). The base medium was composed of a 1:1 mixture of ATCC-formulated Eagle's Minimum Essential Medium, (Cat No. 30-2003), and F12 Medium. The complete growth medium was supplemented with 10% fetal bovine serum, 1× amino acids, 1× sodium bicarbonate, and 1× penicillin-streptomycin (Gibco) and cells incubated at 37° C. in a humidified atmosphere of 5% CO₂. On Day One, SH-SY5Y cells were plated in 96 well fibronectin coated tissue culture plates and allowed to attach overnight. On Day Two, complete media was removed and replaced with RNAi agent in serum free media. Cells were incubated with RNAi agent for 72 hours before analysis of gene expression. Analysis of changes in gene expression in RNAi treated SH-SY5Y cells was measured using Cells-to-C_{T} Kits following the manufacturer's protocol (ThermoFisher A35377). Predesigned gene expression assays (supplied as 20× mixtures) were selected from Applied Bio-systems (Foster City, CA, USA). The efficiencies of these assays (mRNA Target 1 or 2 and ThermoFisher Hs99999905_ml GAPDH) were characterized with a dilution series of cDNA. RT-QPCR was performed in MicroAmp Optical 384-well reaction plates using QuantStudio 7 Flex system. The delta-delta CT method of normalizing to the housekeeping gene GAPDH was used to determine relative amounts of gene expression. GraphPad Prism v9.0 was used to determine IC50 with a four parameter logistic fit.

HEP3B cells (ATCC HB-8064) were cultured in medium containing ATCC-formulated Eagle's Minimum Essential Medium, Catalog No. 30-2003 containing fetal bovine serum at a final concentration of 10%. On Day One, HEP3B cells were plated in 96 well tissue culture plates and allowed to attach overnight. On Day Two, complete media was removed and replaced with RNAi agent in serum free media. Cells were incubated with RNAi agent for 72 hours before analysis of protein expression. Analysis of changes in protein expression in RNAi treated HEP3B cells was measured using a Perkin Elmer AlphaLISA assay for evaluating protein levels corresponding to expression of mRNA Target 1 (AL395) according to manufacturer's protocol. GraphPad Prism v9.0 was used to determine IC50 with a four parameter logistic fit.

Astrocytes derived from iPSCs (Cellular Dynamics 01434) were grown according to manufacturer's protocol. One Day One, astrocytes were plated in 96 well tissue culture plates and allowed to attach overnight. On Day Two, complete media was removed and replaced with RNAi agent in serum free media. Cells were incubated with RNAi agent for 72 hours before analysis of gene expression using methods described above (Thermofisher assay ID Hs00171168_ml).

Mouse Primary Cortical Neuron (MCN) Culture and RNAi Treatment and Analysis: Mouse primary cortical neurons were isolated from wild type C57BL6 mouse embryos at E18. Cells were plated in poly-D-lysine coated 96-well plates at a density of 40 k cells/well and cultured in NbActiv1 (BrainBits, LLC) containing 1% Antibiotic/Antimycotic (Corning) for 7 days at 37° C. in a tissue culture incubator in a humidified chamber with 5% CO₂. On Day 7, half of the medium was removed from each well and 2× concentration of RNAi in culture media with 2% FBS was added for treatment as CRC and incubated with cells for additional 7, 14 or 21 days. Half media change was done every 7 days with fresh culture media. At the end of RNAi treatment, RT-qPCR was performed to quantify SNCA levels using TaqMan Fast Advanced Cell-to-CT kit. Specifically, cells were lysed, cDNA was generated on Mastercycler X50a (Eppendorf), and qPCR was carried out on QuantStudio 7 Flex Real-Time PCR System (Applied Biosystems). The APOE gene expression levels were normalized by (3-actin (ThermoFisher, Mm02619580_g1) using respective probes.

293T Luciferase Transfection, RNAi Treatment and Analysis: 293T cells transfected with the pMIR-luciferase construct (Invitrogen, Waltham, MA) containing the target SNCA sequence were plated overnight at 37° C.; 5% CO₂. Cells were transfected on day two with siRNAs using RNAiMAX (Invitrogen, Waltham, MA) using the protocol provided by the manufacturer. Cells were incubated at 37° C.; 5% CO2 for 48 hrs. Plates were cooled to room temperature followed by the addition of an equal volume of Bio-Glo (Promega, Madison, WI) to each well. Plates were incubated in the dark at room temperature and read on a BioTek Neos2 plate reader (Agilent, Santa Clara, CA).

Representative results from these analyses are shown in Table 11.

### Results

Tables 8, 9, 10, and 11 show the in vitro activities of the RNAi agents against mRNA Target 1 (APOE) and mRNA Target 2 (SNCA) in SH-SY5Y, HEP3B, astrocyte cells, and mouse primary cortical neurons (MCN).

TABLE 8
In vitro activity of mRNA Target 1 (APOE) double
stranded RNAs in HEP3B and Astrocytes.

| | HEP3B | HEP3B | iAstrocyte | iAstrocyte | Pattern | |
|---|---|---|---|---|---|---|
| dsRNA | protein | protein | mRNA | mRNA | 5′-3′ | 5′-3′ |
| No. | % KD | IC50 nM | % KD | IC50 nM | AntiSense2′F | Sense2′F |
| 1 | 77 | 405 | 91 | 101 | 2, 6, 14, 16 | 7, 9, 10, 11 |
| 1 | 84 | 180 | 97 | 45 | 2, 5, 14, 16 | 7, 9, 10, 11 |
| 1 | 73 | 3266 | 96 | 89 | 2, 4, 14, 16 | 7, 9, 10, 11 |
| 1 | 69 | 1168 | 96 | 39 | 2, 7, 14, 16 | 7, 9, 10, 11 |
| 1 | 66 | na | 95 | 59 | 2, 3, 7, 14, 16 | 7, 9, 10, 11 |
| 1 | 87 | 215 | 98 | 44 | 2, 5, 7, 14, 16 | 7, 9, 10, 11 |
| 1 | 92 | 32 | | | 2, 5, 7, 14, 16 | 9, 10, 11 |
| 2 | 93 | 76 | 99 | 28 | 2, 6, 14, 16 | 7, 9, 10, 11 |
| 2 | 92 | 27 | 99 | 19 | 2, 5, 14, 16 | 7, 9, 10, 11 |
| 2 | 93 | 37 | 99 | 15 | 2, 4, 14, 16 | 7, 9, 10, 11 |
| 2 | 94 | 20 | 99 | 13 | 2, 7, 14, 16 | 7, 9, 10, 11 |
| 2 | 92 | 14 | 99 | 13 | 2, 3, 7, 14, 16 | 7, 9, 10, 11 |
| 2 | 91 | 25 | 99 | 13 | 2, 5, 7, 14, 16 | 7, 9, 10, 11 |
| 2 | 94 | 11 | | | 2, 5, 7, 14, 16 | 9, 10, 11 |
| 3 | 80 | 104 | 94 | 64 | 2, 6, 14, 16 | 7, 9, 10, 11 |
| 3 | 83 | 184 | 88 | 71 | 2, 5, 14, 16 | 7, 9, 10, 11 |
| 3 | 76 | 285 | 82 | 219 | 2, 4, 14, 16 | 7, 9, 10, 11 |
| 3 | 84 | 155 | 94 | 6.6 | 2, 7, 14, 16 | 7, 9, 10, 11 |
| 3 | 83 | 54 | 98 | 37 | 2, 3, 7, 14, 16 | 7, 9, 10, 11 |
| 3 | 86 | 115 | 92 | 37 | 2, 5, 7, 14, 16 | 7, 9, 10, 11 |
| 3 | 95 | 21 | | | 2, 5, 7, 14, 16 | 9, 10, 11 |
| 4 | 74 | 135 | 76 | 57 | 2, 6, 14, 16 | 7, 9, 10, 11 |
| 4 | 76 | 203 | 74 | 191 | 2, 5, 14, 16 | 7, 9, 10, 11 |
| 4 | 66 | 287 | 74 | 244 | 2, 4, 14, 16 | 7, 9, 10, 11 |
| 4 | 85 | 75 | 92 | 95 | 2, 7, 14, 16 | 7, 9, 10, 11 |
| 4 | 77 | 159 | 85 | 111 | 2, 3, 7, 14, 16 | 7, 9, 10, 11 |
| 4 | 87 | 71 | 94 | 56 | 2, 5, 7, 14, 16 | 7, 9, 10, 11 |
| 4 | 84 | 10 | | | 2, 5, 7, 14, 16 | 9, 10, 11 |

TABLE 9
In vitro activity of mRNA Target 2 (SNCA)
double stranded RNAs in Sy5y Neurons.

| | |
|---|---|
| Experiment 1 | Experiment 2 |

| dsRNA | Rel IC50 | % pct | Rel IC50 | % pct |
|---|---|---|---|---|
| No. | (nM) | Rel Max | (nM) | Rel Max |
| 6 | 30 | 78.8 | — | — |
| 7 | 117 | 77.4 | 52.5 | 80.1 |
| 7 | 74.9 | 63.4 | 21.7 | 77.3 |
| 7 | 95.3 | 62.8 | 94.7 | 74.7 |
| 7 | 201 | 73.1 | 24 | 74.3 |
| 7 | 43 | 74.2 | 33.1 | 78.4 |
| 7 | 83.8 | 71.4 | 72.8 | 75.2 |
| 7 | 211 | 68 | 43.6 | 81.1 |
| 7 | 243 | 62.6 | 41.3 | 72.7 |
| 7 | 251 | 68.9 | 54.1 | 76 |
| 7 | 51 | 80.1 | — | — |
| 8 | 111 | 69.5 | 62.4 | 71.8 |
| 9 | 46.6 | 73.9 | 25.7 | 79.9 |
| 7 | 329 | 68 | 111 | 82.4 |
| 7 | 203 | 79.2 | 45.7 | 78.1 |
| 7 | 21.5 | 75.5 | — | — |
| 7 | 18.9 | 84.1 | — | — |
| 7 | 135 | 64.8 | 34.2 | 77 |
| 10 | 243.77 | 65.04 | — | — |
| 11 | 332.73 | 67.48 | — | — |
| 12 | 130.99 | 74.95 | — | — |
| 12 | 54.17 | 76.53 | — | — |
| 12 | 144.71 | 77.32 | — | — |
| 12 | 298.64 | 65.78 | — | — |
| 12 | 163.81 | 77.67 | — | — |
| 7 | 165.61 | 73.56 | — | — |
| 12 | 56.26 | 70.94 | — | — |
| 7 | 43.79 | 61.48 | — | — |
| 13 | 37.01 | 65.34 | — | — |
| 14 | 135.64 | 69.17 | — | — |
| 7 | 39.80 | 70.43 | — | — |
| 6 | 229.01 | 68.06 | — | — |
| 6 | 125.75 | 77.99 | — | — |
| 15 | 49.8 | 77.4 | — | — |
| 16 | N/A | N/A | — | — |
| 17 | 84.2 | 74.2 | — | — |
| 18 | 6.39 | 73.3 | — | — |
| 19 | N/A | N/A | — | — |
| 20 | 223 | 64.2 | — | — |
| 21 | N/A | N/A | — | — |
| 22 | N/A | N/A | — | — |
| 23 | N/A | N/A | — | — |
| 24 | N/A | N/A | — | — |
| 25 | 6 | 82.3 | — | — |
| 26 | 34.5 | 77.6 | — | — |
| 27 | 6.09 | 75.9 | — | — |
| 28 | 4.46 | 81.4 | — | — |
| 29 | >1000 | 12 | — | — |
| 30 | 1.26 | 72.40 | — | — |
| 31 | 2.08 | 75.40 | — | — |
| 32 | 0.86 | 78.50 | — | — |
| 33 | 1000.00 | 8.58 | — | — |
| 34 | 55.15 | 72.28 | — | — |
| 35 | 2.06 | 66.30 | — | — |
| 36 | 1.55 | 70.40 | — | — |
| 37 | 1.86 | 79.70 | — | — |
| 38 | 3.73 | 78.70 | — | — |

TABLE 10
In vitro activity of mRNA Target 2 (SNCA) double
stranded RNAs in mouse cortical neurons (MCNs).

| dsRNA | MCN | MCN % KD | MCN % KD |
|---|---|---|---|
| No. | IC50 | at 1 uM | at 4 nM |
| 6 | 2.3 | 99 | 60.7 |
| 7 | 3 | 99.7 | 63.4 |
| 7 | 5.5 | 99.4 | 54.9 |
| 7 | 4.5 | 99.2 | 48.8 |
| 7 | 5 | 99.4 | 66 |
| 7 | 1.86 | 99.7 | 73 |
| 7 | 4.5 | 99.4 | 57.7 |
| 7 | 2.6 | 99.6 | 67.7 |
| 7 | 2.2 | 99.3 | 56.4 |
| 7 | 3 | 98.6 | 50.3 |
| 7 | 1.8 | 99.6 | 72 |
| 8 | 2.1 | 99.3 | 67.5 |
| 9 | 4.8 | 99.6 | 60.7 |
| 7 | 3.5 | 99.6 | 64.5 |
| 7 | 8 | 98.7 | 35 |
| 7 | 1.7 | 99.6 | 70.1 |
| 7 | 2.3 | 99.6 | 65.1 |
| 7 | 3.1 | 99.6 | 63.4 |
| 10 | 4.533 | 97.3 | 38.4 |
| 11 | 6.18 | 96.7 | 28 |
| 12 | 3.116 | 98.3 | 48 |
| 12 | 3.332 | 99 | 48 |
| 12 | 6.607 | 96.8 | 34.5 |
| 12 | 3.11 | 98.8 | 49.8 |
| 12 | 4.079 | 98.4 | 44 |
| 7 | 3.737 | 99 | 50.1 |
| 12 | 3.819 | 98.3 | 44.5 |
| 7 | 3.19 | 98.5 | 47.6 |
| 13 | 3.666 | 98.2 | 40.4 |
| 14 | 5.949 | 97.3 | 31.8 |
| 7 | 3.251 | 98.7 | 47.6 |
| 6 | 10.99 | 97.4 | 10.7 |
| 6 | 3.664 | 99 | 50.3 |
| 15 | 8.4 | 32 | 31.1 |
| 16 | 5.47 | 96.8 | 48.78 |
| 17 | 5.844 | 98.3 | 45.7 |
| 18 | 0.92 | 99.53 | 82.13 |
| 19 | 5.09 | 94.7 | 43.8 |
| 20 | 3.02 | | 57.6 |
| 21 | 3.034 | 92.03 | 48.72 |
| 22 | 41.8 | 58.15 | 1.63 |
| 23 | 10.08 | 85.71 | 34 |
| 24 | 23.55 | 83.34 | 12.65 |
| 25 | 1.94 | 98.93 | 68.16 |
| 26 | 9.41 | 89.35 | 24.16 |
| 27 | 3.22 | 98.12 | 49.57 |
| 28 | 2.96 | 95 | 56.29 |
| 29 | 5.95 | — | 38.4 |

TABLE 11
Luciferase reporter analyses of in vitro activity
of mRNA Target 2 (SNCA) double stranded RNAs.

| | Luciferase | | | |
|---|---|---|---|---|
| ds RNA | Rel IC50 | % KD | % KD | % KD |
| No. | (10 nM) | (50 nM) | (10 nM) | (0.08 nM) |
| 15 | — | — | — | — |
| 16 | 0.0844 | 89.77145726 | — | 42.65963399 |
| 17 | — | — | — | — |
| 18 | — | — | — | — |
| 19 | 0.03691 | — | 82.11 | — |
| 20 | — | — | — | — |
| 21 | 0.0762 | — | 87.29001324 | 44.40367302 |
| 22 | 0.4573 | — | 63.1203076 | 2.780439406 |
| 23 | 0.2901 | — | 79.29184843 | −9.237009968 |
| 24 | 0.0649 | — | 83.19747228 | 46.52584318 |
| 25 | — | — | — | — |
| 26 | — | — | — | — |
| 27 | — | — | — | — |
| 28 | — | — | — | — |
| 29 | 0.09 | — | 50.01 | — |
| 30 | — | — | — | — |
| 31 | — | — | — | — |
| 32 | — | — | — | — |
| 33 | 0.02871 | — | 86.59 | — |
| 34 | — | — | — | — |
| 35 | — | — | — | — |
| 36 | — | — | — | — |
| 37 | — | — | — | — |
| 38 | — | — | — | — |

### Example 3. In Vivo Characterization of Selected RNAi Agents in Mice

The efficacy of the RNAi agents for mRNA Target 1 (APOE) was studied in mRNA Target 1 knock-in (KI) mice in the brain and liver. Six mice received intracerebroventricular (ICV) injection of 100 μg of the RNAi agent (e.g., dsRNAs described in Table 2) or PBS (phosphate buffered saline), and were sacrificed on either at D15, or D57 after the injection.

Human mRNA Target 1 (APOE) expression in spinal cord and brain were measured and analyzed by quantitative PCR (qPCR). Another cohort of mRNA Target 1 (APOE) KI mice received SC injection of the RNAi agent or PBS and were serially bled for up to 12 weeks post injection and serum analyzed for mRNA Target 1(APOE) protein levels as described above.

Representative results are shown in Table 12. Data in Table 12 shows dsRNA constructs comprising 2′F-modifications at positions 2, 5, 7, 14, and 16 of the antisense strand were more durable in vivo than dsRNA constructs containing 2′F-modifications at other positions. Additionally, injection of dsRNA constructs comprising 2′F-modifications at positions 2, 5, 7, 14, and 16 of the antisense strand, at two different concentrations, resulted in reduction of mRNA Target 1(APOE) protein serum concentration in vivo for at least 8 weeks and reduction of mRNA Target 1 (APOE) expression in several types of brain tissue, including brain stem (BS), hippocampus (H), hippocampal cortex (HCTX), and frontal cortex (FCTX).

The efficacy of the RNAi agents for mRNA Target 2 (SNCA) was studied in wildtype C56BL/6N mice. Six mice received intracerebroventricular (ICV) injection of 30 μg of the RNAi agent (e.g., dsRNA described in Table 4) or PBS (phosphate buffered saline), and were sacrificed on either at D15, or D57 after the injection. Mouse mRNA Target 2 (SNCA) mRNA expression in spinal cord and brain were measured and analyzed by quantitative PCR (qPCR). Data are shown in Table 13 and indicate high levels of mRNA Target 2 (SNCA) knockdown at 2 months following ICV injection. The highest levels of mRNA Target 2 (SNCA) knockdown were observed in the brain stem, frontal cortex, striatum (STR), and lumbar spinal cord tissues (e.g., LDRG cells) of mouse subjects (see Table 13).

TABLE 12
In vivo activity of mRNA Target 1 (APOE).

| | | | | Days | | Pattern | |
|---|---|---|---|---|---|---|---|
| dsRNA | | | | post | Percent | 5′-3′ | 5′-3′ |
| No. | Tissue/Brain Region | dose/route | | dose | Remaining | AntiSense2′F | Sense2′F |
| 2 | Lumber Spinal Cord | 100 | ug/ICV | 15 | 5 | 2, 5, 7, 14, 16 | 7, 9, 10, 11 |
| 2 | Hippocampus | 100 | ug/ICV | 15 | 24 | 2, 5, 7, 14, 16 | 7, 9, 10, 11 |
| 2 | Frontal Cortex | 100 | ug/ICV | 15 | 36 | 2, 5, 7, 14, 16 | 7, 9, 10, 11 |
| 2 | Brain Stem | 100 | ug/ICV | 15 | 3 | 2, 5, 7, 14, 16 | 7, 9, 10, 11 |
| 2 | Liver | 100 | ug/ICV | 15 | 41 | 2, 5, 7, 14, 16 | 7, 9, 10, 11 |
| 2 | Liver | 10 | mg/kg/IV | 7 | 29 | 2, 5, 7, 14, 16 | 9, 10, 11 |
| 2 | Lumber Spinal Cord | 100 | ug/ICV | 15 | 4 | 2, 5, 7, 14, 16 | 7, 9, 10, 11 |
| 2 | Hippocampus | 100 | ug/ICV | 15 | 35 | 2, 5, 7, 14, 16 | 7, 9, 10, 11 |
| 2 | Frontal Cortex | 100 | ug/ICV | 15 | 57 | 2, 5, 7, 14, 16 | 7, 9, 10, 11 |
| 2 | Brain Stem | 100 | ug/ICV | 15 | 3 | 2, 5, 7, 14, 16 | 7, 9, 10, 11 |
| 2 | Liver | 100 | ug/ICV | 15 | 67 | 2, 5, 7, 14, 16 | 7, 9, 10, 11 |
| 2 | Liver | 10 | mg/kg/IV | 7 | 28 | 2, 5, 7, 14, 16 | 9, 10, 11 |
| 2 | Lumber Spinal Cord | 100 | ug/ICV | 57 | 14 | 2, 5, 7, 14, 16 | 7, 9, 10, 11 |
| 2 | Hippocampus | 100 | ug/ICV | 57 | 51 | 2, 5, 7, 14, 16 | 7, 9, 10, 11 |
| 2 | Frontal Cortex | 100 | ug/ICV | 57 | 77 | 2, 5, 7, 14, 16 | 7, 9, 10, 11 |
| 2 | Brain Stem | 100 | ug/ICV | 57 | 25 | 2, 5, 7, 14, 16 | 7, 9, 10, 11 |
| 2 | Liver | 100 | ug/ICV | 57 | 92 | 2, 5, 7, 14, 16 | 7, 9, 10, 11 |
| 2 | Liver | 10 | mg/kg/IV | 57 | 44 | 2, 5, 7, 14, 16 | 9, 10, 11 |
| 2 | Lumber Spinal Cord | 100 | ug/ICV | 57 | 22 | 2, 5, 7, 14, 16 | 7, 9, 10, 11 |
| 2 | Hippocampus | 100 | ug/ICV | 57 | 61 | 2, 5, 7, 14, 16 | 7, 9, 10, 11 |
| 2 | Frontal Cortex | 100 | ug/ICV | 57 | 84 | 2, 5, 7, 14, 16 | 7, 9, 10, 11 |
| 2 | Brain Stem | 100 | ug/ICV | 57 | 50 | 2, 5, 7, 14, 16 | 7, 9, 10, 11 |
| 2 | Liver | 100 | ug/ICV | 57 | 99 | 2, 5, 7, 14, 16 | 7, 9, 10, 11 |
| 2 | Liver | 10 | mg/kg/IV | 57 | 47 | 2, 5, 7, 14, 16 | 9, 10, 11 |

TABLE 13
In vivo activity of mRNA Target
2 (SNCA) double stranded RNAs.

| | Percent Knockdown | | | | |
|---|---|---|---|---|---|
| | | | | | Lumbar |
| | | | | | Dorsal |
| | | | | Lumbar | Root |
| dsRNA | Brain | Frontal | Striatum | Spinal | Ganglion |
| No. | Stem | Cortex | (STR) | Cord | (LDRGs) |
| 6 | 79 | 63 | 70 | 42 | 16 |
| 7 | 83 | 52 | 60 | 70 | 16 |
| 7 | 72 | 51 | 53 | 28 | 10 |
| 7 | 79 | 48 | 61 | 45 | 17 |
| 7 | 72 | 47 | 43 | 43 | 22 |
| 7 | 73 | 46 | 57 | 42 | 16 |
| 7 | 78 | 43 | 59 | 30 | 16 |
| 7 | 69 | 43 | 56 | 51 | 14 |
| 7 | 75 | 42 | 46 | 43 | 17 |
| 7 | 70 | 40 | 40 | 52 | 24 |
| 7 | 77 | 39 | 60 | 29 | 18 |
| 8 | 63 | 38 | 42 | 11 | 11 |
| 9 | 52 | 34 | 49 | 41 | 12 |
| 7 | 59 | 33 | 49 | 40 | 18 |
| 7 | 59 | 33 | 34 | 24 | 22 |
| 7 | 78 | 31 | 30 | 7 | 14 |
| 7 | 75 | 28 | 64 | 38 | 27 |
| 7 | 67 | 25 | 39 | 36 | 3 |
| 10 | 81 | 60 | 59 | 64 | 26 |
| 11 | 74 | 55 | 67 | 60 | 17 |
| 12 | 52 | 31 | 22 | 36 | 16 |
| 12 | 79 | 65 | 64 | 61 | 28 |
| 12 | 75 | 59 | 43 | 62 | 41 |
| 12 | 74 | 52 | 62 | 46 | 29 |
| 12 | 74 | 52 | 63 | 58 | 40 |
| 7 | 73 | 57 | 65 | 34 | 10 |
| 12 | 55 | 47 | 44 | 45 | 25 |
| 7 | 82 | 50 | 63 | 50 | 23 |
| 13 | 71 | 52 | 68 | 41 | 40 |
| 14 | 60 | 40 | 47 | 53 | 42 |
| 7 | 74 | 44 | 66 | 56 | 27 |
| 6 | 62 | 45 | 50 | 45 | 32 |
| 6 | 79 | 58 | 72 | 71 | 33 |

### Example 4. siRNA Conjugates

This example describes production of double stranded nucleic acid-GalNAc conjugates. For the synthesis of GalNAc-conjugated sense strands, a sense strand with a 3′ C6-NH2 functional group is first synthesized using standard phosphoramidite chemistry. A stock solution of GalNAc ligand-NHS ester (10 mmol/L in acetonitrile; 1 eq) is prepared. Borate buffer (10% v/v; 20×) is added to oligonucleotide C6-NH2 sense strand in an Eppendorf tube, then GalNAc ligand (5 eq) is added. The mixture is shaken at ambient temperature for 16 hours. After this time, the mixture is transferred to a 15 mL falcon tube, ammonium hydroxide (28 mass %) is added, and the mixture is shaken at ambient temperature for 2 hours. The ammonia is then removed in vacuo. The residue is purified by ion-exchange chromatography. Conditions: Solvent A: 15% MeCN/20 mM NaH2PO4, Solvent B: 15% MeCN/20 mM NaH2PO4, 1M NaBr; 35-55% B over 5 CV at 8 mL/min, column temperature 60° C. The desired fractions are pooled and desalted by spin-filtration using an Eppendorf centrifuge or desalting column. After desalting, the material is recovered and OD and volume are measured to obtain concentration.

Alternatively, conjugation is to the 5′ position of the sense strand through immobilizing the GalNAc ligand on microporous polystyrene resin or controlled pore glass and synthesizing using established solid phase oligonucleotide synthesis methods with 5′-CE β-cyanoethyl) phosphoramidites.

Alternatively, the GalNAc ligand is converted to a suitable phosphoramidite and delivered to the 5′ position of the sense strand using standard phosphoramidite chemistry.

To generate the siRNA duplexes of a sense and antisense strand, the following procedures are performed. To a falcon tube containing oligonucleotide sense strand-GalNAc conjugate, the corresponding antisense oligonucleotide (1 eq) is added and vortexed for 10 seconds before spin-filtering through 100K MWCO Amicon filter unit to remove particulates.

The filtrate is recovered and concentrated in vacuo on a Genevac evaporator. The residue is reconstituted in 1×PBS, filtered through 0.2μ filter, and OD and volume are measured to obtain concentration.

An endotoxin test is performed using a Limulus amebocyte lysate on an Endosafe® NexGen PTS instrument.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A double stranded nucleic acid comprising:
(i) a sense strand; and
(ii) an antisense strand having a 5′-end and a 3′-end, and consisting of modified nucleotides, wherein the modified nucleotides do not comprise more than five 2′-fluoro (2′F)-modified nucleotides, and wherein the 2′F-modified nucleotides are located at positions 2, 5, 7, 14, and 16 relative to the 5′-end of the antisense strand.

2. A double stranded nucleic acid comprising:
(i) a sense strand; and
(ii) an antisense strand having a 5′-end and a 3′-end, and consisting of modified nucleotides, wherein the modified nucleotides do not comprise more than five 2′F-modified nucleotides, and wherein the 2′F-modified nucleotides are located at positions 2, 3, 7, 14, and 16 relative to the 5′-end of the antisense strand.

3. A double stranded nucleic acid comprising:
(i) a sense strand; and
(ii) an antisense strand having a 5′-end and a 3′-end, and consisting of modified nucleotides, wherein the modified nucleotides do not comprise more than five 2′F-modified nucleotides, and wherein the 2′F-modified nucleotides are located at positions 2, 5, 8, 14, and 16 relative to the 5′-end of the antisense strand.

4. A double stranded nucleic acid comprising:
(i) a sense strand; and
(ii) an antisense strand having a 5′-end and a 3′-end, and consisting of modified nucleotides, wherein the modified nucleotides do not comprise more than six 2′F-modified nucleotides, and wherein the 2′F-modified nucleotides are located at positions 2, 4, 6, 8, 14, and 16 relative to the 5′-end of the antisense strand.

5. A double stranded nucleic acid comprising:
(i) a sense strand; and
(ii) an antisense strand having a 5′-end and a 3′-end, and consisting of modified nucleotides, wherein the modified nucleotides do not comprise more than five 2′F-modified nucleotides, and wherein the 2′F-modified nucleotides are located at positions 2, 6, 8, 14, and 16 relative to the 5′-end of the antisense strand.

6. The double stranded nucleic acid of any one of claims 1 to 5, wherein the sense strand comprises a 5′-end and a 3′-end, and comprises 2′F-modified nucleotides at:
(i) positions 9, 10, and 11 relative to the 5′-end of the sense strand;
(ii) positions 7, 9, and 11 relative to the 5′-end of the sense strand;
(iii) positions 7, 9, and 10 relative to the 5′-end of the sense strand; or
(iv) positions 7, 10, and 11 relative to the 5′-end of the sense strand.

7. The double stranded nucleic acid of claim 6, wherein the sense strand does not comprise any other 2′F-modified nucleotides.

8. A double stranded nucleic acid comprising a sense strand and an antisense strand, wherein the sense strand and the antisense strand form a duplex, wherein the antisense strand comprises a sequence complementary to a portion of a target mRNA, and wherein the antisense strand comprises five 2′F-modified nucleotides at one of the following set of positions from the 5′ end of the antisense strand: (a) positions 2, 5, 7, 14, and 16; (b) positions 2, 3, 7, 14, and 16; (c) positions 2, 5, 8, 14, and 16, and no other 2′F-modified nucleotides.

9. The double stranded nucleic acid of claim 8, wherein the sense strand comprises three 2′F-modified nucleotides at one of the following set of positions from the 5′ end of the sense strand: (a) positions 9, 10, and 11; (b) positions 7, 9, and 11; (c) positions 7, 9, 10; or (d) positions 7, 10, and 11.

10. The double stranded nucleic acid of claim 8 or 9, wherein the antisense strand comprises 2′-O-methyl modified nucleotides at positions other than the 2′F-modified positions.

11. The double stranded nucleic acid of any one of claims 8 to 10, wherein the sense strand comprises 2′-O-methyl modified nucleotides, 2′-O—C₁₂₋₁₆ alkyl modified nucleotides, or optionally one or more abasic moieties at positions other than the 2′F-modified positions.

12. The double stranded nucleic acid of any one of claims 1 to 11, wherein position 1 relative to the 5′-end of the sense strand or 5′-end of the antisense strand comprises a 5′ phosphate analog.

13. The double stranded nucleic acid of claim 12, wherein the 5′ phosphate analog comprises a 5′ vinyl phosphonate group.

14. The double stranded nucleic acid of claim 12 or 13, wherein the antisense strand comprises the 5′ phosphate analog.

15. The double stranded nucleic acid of any one of claims 1 to 14, wherein the sense strand is between 18 and 24 nucleotides in length.

16. The double stranded nucleic acid of any one of claims 1 to 15, wherein the sense strand is 21 nucleotides in length.

17. The double stranded nucleic acid of any one of claims 1 to 16, wherein the antisense strand is between 18 and 24 nucleotides in length.

18. The double stranded nucleic acid of any one of claims 1 to 17, wherein the antisense strand is 23 nucleotides in length.

19. The double stranded nucleic acid of any one of claims 1 to 18, wherein the sense strand and the antisense strand do not have the same length.

20. The double stranded nucleic acid of any one of claims 1 to 19, wherein the antisense strand is longer than the sense strand, optionally wherein the antisense strand is between 2 and 10 nucleotides longer than the sense strand.

21. The double stranded nucleic acid of any one of claims 1 to 20, wherein the modified nucleotides are modified ribonucleotides.

22. The double stranded nucleic acid of any one of claims 1 to 21, wherein the modified nucleotides of the sense strand comprise one or more 2′-O-methyl (2′OMe)-modified nucleotides or one or more 2′-O— C₁₂₋₁₆ alkyl modified nucleotides.

23. The double stranded nucleic acid of any one of claims 1 to 22 comprising one or more abasic moieties.

24. The double stranded nucleic acid of any one of claims 1 to 23, wherein the modified nucleotides of the sense strand comprise only 2′OMe-modified nucleotides with the exception of the 2′-fluoro (2′F)-modified nucleotides at the listed positions.

25. The double stranded nucleic acid of any one of claims 1 to 23, wherein the modified nucleotides of the sense strand comprise one or more 2′-O-methyl (2′OMe)-modified nucleotides, one or more 2′-O— C₁₂₋₁₆ alkyl modified nucleotides, or one or more abasic moieties.

26. The double stranded nucleic acid of any one of claims 1 to 25, wherein the modified nucleotides of the antisense strand comprise only 2′OMe-modified nucleotides with the exception of the 2′-fluoro (2′F)-modified nucleotides at the listed positions.

27. The double stranded nucleic acid of any one of claims 1 to 26, wherein the sense strand comprises one or more modified internucleotide linkages.

28. The double stranded nucleic acid of claim 27, wherein the sense strand comprises four modified internucleotide linkages.

29. The double stranded nucleic acid of claim 27 or 28, wherein the modified internucleotide linkages comprise one or more phosphorothioate (PS) internucleotide linkages.

30. The double stranded nucleic acid of claim 29, wherein each of the modified internucleotide linkages is a PS internucleotide linkage.

31. The double stranded nucleic acid of any one of claims 1 to 30, wherein the antisense strand comprises one or more modified internucleotide linkages.

32. The double stranded nucleic acid of claim 31, wherein the antisense strand comprises four modified internucleotide linkages.

33. The double stranded nucleic acid of claim 31 or 32, wherein the modified internucleotide linkages comprise one or more phosphorothioate (PS) internucleotide linkages.

34. The double stranded nucleic acid of claim 33, wherein each of the modified internucleotide linkages is a PS internucleotide linkage.

35. The double stranded nucleic acid of any one of claims 27 to 34, wherein positions 1 and 2 relative to the 5′ end of the sense strand are linked by a modified internucleotide linkage, optionally a phosphorothioate (PS) internucleotide linkage.

36. The double stranded nucleic acid of any one of claims 27 to 35, wherein positions 1 and 2 relative to the 5′ end of the antisense strand are linked by a modified internucleotide linkage, optionally a phosphorothioate (PS) internucleotide linkage.

37. The double stranded nucleic acid of any one of claims 27 to 36, wherein positions 2 and 3 relative to the 5′ end of the sense strand are linked by a modified internucleotide linkage, optionally a phosphorothioate (PS) internucleotide linkage.

38. The double stranded nucleic acid of any one of claims 27 to 37, wherein positions 2 and 3 relative to the 5′ end of the antisense strand are linked by a modified internucleotide linkage, optionally a phosphorothioate (PS) internucleotide linkage.

39. The double stranded nucleic acid of any one of claims 27 to 38, wherein at least two of positions 1, 2, and 3 of the 3′ end of the sense strand are linked by a modified internucleotide linkage, optionally a phosphorothioate (PS) internucleotide linkage.

40. The double stranded nucleic acid of any one of claims 27 to 39, wherein at least two of positions 1, 2, and 3 of the 3′ end of the antisense strand are linked by a modified internucleotide linkage, optionally a phosphorothioate (PS) internucleotide linkage.

41. The double stranded nucleic acid of any one of claims 27 to 40, wherein each of positions 1, 2, and 3 relative to the 3′ end of the sense strand are linked by a modified internucleotide linkage, optionally a phosphorothioate (PS) internucleotide linkage.

42. The double stranded nucleic acid of any one of claims 27 to 41, wherein each of positions 1, 2, and 3 relative to the 3′ end of the antisense strand are linked by a modified internucleotide linkage, optionally a phosphorothioate (PS) internucleotide linkage.

43. A conjugate comprising the double stranded nucleic acid of any one of claims 1 to 42.

44. The conjugate of claim 43, wherein the conjugate comprises a structure of Formula I, wherein Formula I comprises:
A-B-C Formula I,
wherein “A” of Formula I comprises the double-stranded nucleic acid, “B” of Formula I comprises a bond or a linker, and “C” of Formula I comprises a delivery molecule.

45. The conjugate of claim 44, wherein “B” of Formula I is connected to the 5′ end or the 3′ end of the sense strand of the double stranded nucleic acid.

46. The conjugate of claim 44 or 45, wherein “B” of Formula I is connected to the 3′ end of the sense strand of the double stranded nucleic acid.

47. The conjugate of claim 44, wherein “B” of Formula I is connected to the 5′ end or the 3′ end of the antisense strand of the double stranded nucleic acid.

48. The conjugate of any one of claims 44 to 47, wherein “B” of Formula I comprises a triethylene glycol (TEG) linker.

49. The conjugate of any one of claims 44 to 48, wherein “C” of Formula I comprises cholesterol.

50. The conjugate of any one of claims 44 to 48, wherein “C” of Formula I comprises tocopherol.

51. The conjugate of any one of claims 43 to 50, wherein the double stranded RNA does not comprise a nucleotide which is connected to: a maleimide group; a tertiary amide which is bonded to a gem-dimethyl (GDM) group; or a C₆—NH₂ group.

52. The conjugate of any one of claims 43 to 51, wherein “B” of Formula I does not comprise: a maleimide-methyl-tetrazine-trans-cyclo-octene (mal-tet-TCO) linker; a succinimidyl-4-(N-maleimidomethyl)cyclohexane-1-carboxylate (SMCC) linker; a linker comprising a tertiary amide which is bonded to a gem-dimethyl (GDM) group; or a linker comprising a C₆—NH₂ group.

53. The conjugate of any one of claims 43 to 47, wherein “B” of Formula I comprises a linker comprising a C₆—NH₂ group.

54. The conjugate of any one of claims 43 to 47, wherein “C” of Formula I comprises one or more N-acetylgalactosamine (GalNAc) moieties.

55. A pharmaceutical composition comprising the double stranded nucleic acid of any one of claims 1 to 42, or the conjugate of any one of claims 43 to 54, and a pharmaceutically acceptable carrier.

56. A method of inhibiting or reducing a target mRNA in a cell, the method comprising contacting the cell comprising the target mRNA with the double stranded nucleic acid of any one of claims 1 to 42, or the conjugate of any one of claims 43 to 54, or the pharmaceutical composition of claim 55.

57. The method of claim 56, wherein the cell is a mammalian cell, optionally a human cell.

58. The method of claim 56 or 57, wherein the cell is in a subject, optionally a human subject.

59. A double stranded nucleic acid comprising a sense strand comprising a sequence set forth in any one of Tables 1-5, and an antisense strand comprising a sequence set forth in any one of Tables 1-5.

60. The double stranded nucleic acid of claim 59, wherein
(i) the sense strand comprises the sequence set forth in any one of SEQ ID NOs: 1, 2, 3, 4, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 77, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 145, 146, 152, 153, 154, 155, 156, 157, 158, 159, 160, 161, 162, 163, 164, 165, 166, 167, 168, 169, 170, 171, 172, 173, 174, or 175; and/or
(ii) the antisense strand comprises the sequence set forth in any one of SEQ ID NOs: 5, 6, 7, 8, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 78, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 147, 148, 149, 150, 151, 176, 177, 178, 179, 180, 181, 182, 183, 184, 185, 186, 187, 188, 189, 190, 191, 192, 193, 194, 195, 196, 197, 198, or 199.
